SEQUENCE LISTING (8 sequences)

SEQ ID NO: 1 - DNA, 24 bp, artificial
  Primer
catgacacccccactgtcgtgtcc

SEQ ID NO: 2 - DNA, 24 bp, artificial
  Primer
tggcaccacagaaatgacctccac

SEQ ID NO: 3 - DNA, 24 bp, artificial
  Primer
tttggtgcacggtcagcagattgg

SEQ ID NO: 4 - DNA, 20 bp, artificial
  Primer
gaacaacatctgcctggagt

SEQ ID NO: 5 - DNA, 21 bp, artificial
  Primer
gtatgacttctgccatgtagc

SEQ ID NO: 6 - DNA, 19 bp, artificial
  Primer
caacagcactgctgtagca

SEQ ID NO: 7 - DNA, 19 bp, artificial
  Primer
ggtgactcagaccttcagc

SEQ ID NO: 8 - DNA, 26 bp, artificial
  Primer
cacaaatcaaggatgaccaaactgag

(19) United States
(12) Patent Application Publication
(10) Pub. No.: US 2016/0195544 A1
(43) Pub. Date: Jul. 7, 2016

(54) METHOD OF DIAGNOSING SEPSIS OR SEPSIS RISK

(71) Applicant: Minsoo KIM, Pittsford, NY (US)

(72) Inventor: Minsoo KIM, Pittsford, NY (US)

(73) Assignee: UNIVERSITY OF ROCHESTER, Rochester, NY (US)

(21) Appl. No.: 14/910,962

(22) PCT Filed: Aug. 6, 2014
(86) PCT No.: PCT/US14/49955

§ 371 Date: Feb. 8, 2016

Related U.S. Application Data
(60) Provisional application No. 61/863,266, filed on Aug. 7, 2013.

(51) Int. Cl. G01N 33/68

(52) U.S. Cl. CPC G01N 33/6893; G01N 2800/52; G01N 2800/24; G01N 2333/7055

(57) ABSTRACT

The present invention relates to methods of diagnosing sepsis or sepsis risk in a subject and methods of treating a patient for sepsis. The methods include detecting the presence of a subpopulation of neutrophils having an elevated integrin VLA-3 (CD49c/CD29) expression level in the subject. The method also includes contacting a biological sample from a subject with a reagent that binds specifically to integrin VLA-3 (CD49c/CD29) in the biological sample, detecting the reagent bound to integrin VLA-3, and determining the expression level of integrin VLA-3 in the sample. The invention further relates to a method of discriminating between sepsis and systemic inflammatory response syndrome (SIRS) and a method of treating a patient for SIRS which includes detecting the presence or absence of a subpopulation of neutrophils having an elevated integrin VLA-3 expression level in a subject having systemic inflammation.

This application claims the benefit of U.S. Provisional Patent Application Ser. No. 61/863,266, filed Aug. 7, 2013, the disclosure of which is incorporated herein by reference in its entirety.

This invention was made with government support under HL087088, HL018208, and HL094797, each of which is awarded by the National Institutes of Health. The government has certain rights in the invention.

### TECHNOLOGICAL FIELD

Disclosed are methods of diagnosing sepsis or sepsis risk, and methods of treating a patient for sepsis. Also disclosed are methods of discriminating between sepsis and systemic inflammatory response syndrome (SIRS), and methods of treating a patient for SIRS.

### BACKGROUND

Severe sepsis, a systemic inflammatory response to infections associated with acute organ dysfunction, is an increasing cause of morbidity and mortality among children and adults, and has been one of the most significant challenges in critical care affecting 750,000 individuals/year with a mortality rate of approximately 30% (Rittirsch et al., “Harmful Molecular Mechanisms in Sepsis,” Nat. Rev. Immunol. 8:776-787 (2008); Angus et al., “Epidemiology of Severe Sepsis in the United States: Analysis of Incidence, Outcome, and Associated Costs of Care,” Crit. Care Med. 29:1303-1310 (2001)). Pro-inflammatory signals arise at the early stage of sepsis and allow circulating neutrophils to access sites of inflammation and to phagocytose foreign pathogens and necrotic/apoptotic cells. Proteolytic enzymes, such as elastase and myeloperoxidase (MPO), are stored in the azurophilic granules within neutrophils and are released in response to a variety of mechanical, thermal, and chemical stimuli at the infected sites. Neutrophils also produce reactive oxygen species such as hydrogen peroxide, superoxide, and nitric oxide (Nathan, C., “Neutrophils and Immunity: Challenges and Opportunities,” Nat. Rev. Immunol. 6:173-182 (2006)). Although these inflammatory mediators are important for the host defense, they also participate in endothelial and extravascular host tissue damage (Nathan, C., “Neutrophils and Immunity: Challenges and Opportunities,” Nat. Rev. Immunol. 6:173-182 (2006)). Thus, in uncontrolled inflammatory conditions such as sepsis, during which many neutrophils become activated at the endothelial interface and in the underlying tissue, excessive inflammatory activities lead to further microvascular dysfunction and tissue damage (Brown et al., “Neutrophils in Development of Multiple Organ Failure in Sepsis,” Lancet 368:157-169 (2006)). Many reports have documented that the sequestration of hyperactive neutrophils in the microvasculature of visceral organs is a major contributing factor in multi-organ failure, which is an important hallmark of sepsis (Brown et al., “Neutrophils in Development of Multiple Organ Failure in Sepsis,” Lancet 368:157-169 (2006); Suda et al., “Neutrophil Elastase Inhibitor Improves Survival of Rats With Clinically Relevant Sepsis,” Shock 33: 526-531 (2010); Kovach et al., “The Function of Neutrophils in Sepsis,” Curr. Opin. Infect. Dis. 25(3):321-7 (2012)). Therefore, neutrophil recruitment could act as a double-edged sword in sepsis.

Therapeutic intervention demands mechanistic knowledge of the leukocyte trafficking step during the systemic inflammatory condition. What are also needed are an accurate means for diagnosing sepsis, discriminating sepsis from systemic inflammatory response syndrome, and treating sepsis. This application is directed to overcoming these and other deficiencies in the art.

### SUMMARY

One aspect relates to a method of diagnosing sepsis or sepsis risk in a subject. The method includes detecting the presence of a subpopulation of neutrophils having an elevated integrin VLA-3 (CD49c/CD29) expression level in the subject, whereby the presence of the subpopulation of neutrophils indicates that the subject has sepsis or a risk of sepsis.

A second aspect relates to a method of diagnosing sepsis or sepsis risk in a subject. The method includes contacting a biological sample from a subject with a reagent that binds specifically to integrin VLA-3 (CD49c/CD29) in the biological sample; detecting the reagent bound to integrin VLA-3 in the biological sample; and determining the expression level of integrin VLA-3 in the biological sample wherein an elevated level of integrin VLA-3 in the biological sample, relative to a control level of integrin VLA-3, indicates that the subject has sepsis or is at risk of developing sepsis.

A third aspect relates to a method of discriminating between sepsis and systemic inflammatory response syndrome (SIRS). The method includes detecting the presence or absence of a subpopulation of neutrophils having an elevated integrin VLA-3 expression level in a subject having systemic inflammation, whereby the presence of the subpopulation of neutrophils indicates that the subject has sepsis and the absence of the subpopulation of neutrophils indicates that that subject has SIRS.

A fourth aspect relates to a method of a method of discriminating between sepsis and systemic inflammatory response syndrome (SIRS). The method includes contacting a biological sample from a subject with a reagent that binds specifically to integrin VLA-3 (CD49c/CD29) in the biological sample; detecting the reagent bound to integrin VLA-3 in the biological sample; and determining the expression level of integrin VLA-3 in the biological sample wherein an elevated level of integrin VLA-3 in the biological sample, relative to a control level of integrin VLA-3, indicates that the subject has sepsis and absence of an elevated level of integrin VLA-3 in the biological sample, relative to a control level of integrin VLA-3, indicates that that subject has SIRS.

Migration of neutrophils to infection sites is vital for pathogen clearance and, thus, host survival. Interaction of cell surface integrins with their counterpart ligands results in the adherence of circulating neutrophils and directed migration to sites of infection. Therefore, an important function of integrins is to concentrate activated neutrophils at the infection site, ensuring that their immune products and activities remain at this site. However, excessive integrin activity during sepsis leads to a massive neutrophil infiltration into non-infected tissues and exaggerated inflammatory response with associated tissue damage.

The accompanying Examples show that, unlike other extracellular matrix binding integrins, integrin VLA-3 (CD49c/CD29) is dramatically upregulated on a subpopulation of neutrophils isolated from both human septic patients and mouse sepsis models. Compared with the Gr1^{high}CD11b^{high}VLA-3^{low} granulocyte population, Gr1^{high}CD11b^{high}VLA-3^{high} cells from septic animals displayed hyper-inflammatory phenotypes. Administration of VLA-3 antagonist peptides and conditional depletion of VLA-3 expression in granulocytes significantly reduced the number of extravasating neutrophils and improved survival in septic mice. Thus, the results presented in the accompanying Examples show that VLA-3 is a unique marker of tissue homing and hyper-responsive neutrophil subtypes in sepsis, and blocking of VLA-3 represents a new therapeutic approach for the treatment of sepsis by selectively targeting the hyper-inflammatory, rapid tissue-infiltrating neutrophil subtype (Gr1^{high} CD11b^{high}VLA-3^{high}). By using multiphoton intravital microscopy (MP-IVM) it is shown that extravasation of neutrophils was significantly reduced by administration of VLA-3 antagonist peptide. Therefore, selective targeting of activated, hyper-inflammatory, rapidly tissue-infiltrating neutrophil subtype (Gr1^{high}CD11b^{high}VLA-3^{high}) via VLA-3 should minimize organ failure during sepsis. The naïve cells are left untouched, thereby circumventing iatrogenic immune suppression, a major problem encountered during other anti-inflammatory therapies.

### BRIEF DESCRIPTION OF THE DRAWINGS

FIGS. 1A-1C demonstrate that integrin VLA-3 is up-regulated on human neutrophils in sepsis. In FIG. 1A, neutrophils from severe non-infectious SIRS patients (n=8, Age 63.5±8.9, Conditions: COPD, lung fibrosis, respiratory distress, squamus cell carcinoma, APCAHE II Score: 21±6), severe sepsis patients (n=6, Age 53.5±15, source of infections: renal, lung, abdomen, genitourinary, Positive blood cultures: 83%, APCAHE II Score: 33±12), and healthy donors (n=5, Age 41±14) were isolated and surface expression of integrins was measured by flow cytometry. The results were expressed as the ratio of the integrin expression Mean Fluorescence Intensity (MFI) to the isotype control MFI of the same donor. *p<0.05. FIG. 1B shows histograms depicting the intensity of VLA-3 expression at different time points following diagnosis. The graph represents one of the six sepsis patients. FIG. 1C shows that neutrophils isolated from healthy donors were stimulated with PMA (20 ng/ml), TNF-α (20 ng/ml), LPS (100 μg/ml) or fMLP (1 μM) for 1 hour or 3 hours. The fold changes in gene expression of VLA-3, compared with that of unstimulated cells was determined by RT-PCR (upper panel), and its surface expression was measured by flow cytometry (bottom panel). The results represent the mean±SEM of 3 separate donors. *p<0.05.

FIG. 2 shows that integrin VLA-3 is up-regulated in murine sepsis. Integrin expression on septic mouse neutrophils was measured by flow cytometry. Cells were isolated from the bone marrow (BM), peritoneal lavage (PL), and peripheral blood of naïve and septic mice at the indicated time points. For the MFI analysis, cells were gated on Gr1^{high} granulocytes. The results were expressed as the fold increases in the MFI over the naïve control (naïve blood MFI was used to calculate the % change in PL expression). The results represent the mean±SEM of four separate animals/time point.

FIGS. 3A-3C show the phenotype of Gr1^{high}CD11b VLA-3^{high} neutrophils. In FIG. 3A, the pseudo color plots show the presence of VLA-3^{high} and VLA-3^{low} populations among the neutrophils isolated from healthy donors, SIRS and Sepsis patients. In FIG. 3B, cells were isolated from PL (12 hours after LPS administration) and stained with CD11b and Gr1 antibodies. The cells were sorted into Gr1^{high} and Gr1^{low} granulocytes and stained with H & E following cytospin. Gr1^{high} cells show neutrophil morphology with a ring or multi lobed nucleus, and Gr1^{low} cells have a monocyte-like appearance. The pseudo color plots show the presence of VLA-3^{high} and VLA-3^{low} populations among the Gr1^{high}CD11b^{high} mature neutrophils in the bone marrow of septic mice. In FIG. 3C, BM cells from naïve and CLP-induced septic mice were isolated at 12 hours and 24 hours following sepsis and sorted into VLA-3^{high} and VLA-3^{low} cells after gating on Gr1^{high}CD11b^{high} granulocytes (Upper panel). The lower panel shows the confirmation of the presence of VLA-3^{high} and VLA-3^{low} cells among neutrophils in BM. Cells were stained for α₃β₁ (PE) and Ly6G (APC) or Gr1 (FITC). Flow cytometry plots show the presence of α₃β₁^{high} and α₃β₁^{low} granulocyte populations.

FIGS. 4A-4B show that Gr1^{high}CD11b^{high}VLA-3^{high} neutrophils have pro-inflammatory phenotypes. In FIG. 4A, cells were sorted as described in FIG. 3C and the differences in IL-6, TNF-α and IL-1β gene expressions were quantified by RT-PCR. The bar graphs shows fold change compared with naïve cells. In FIG. 4B, the myeloperoxidase enzyme (MPO) activity of VLA-3^{high} and VLA-3^{low} cells isolated from the BM of CLP and endotoxemic mice was measured using a bioluminescence assay. Sorted cells (5×10⁴ cells/well) were stimulated with PMA (1 μM) in the presence of luminol (1 mg/well), and the luminescence intensity was imaged. The line graphs (left) show the kinetics of MPO activity in VLA-3^{high} and VLA-3^{low} cells and the respective luminescence images are shown (right).

FIGS. 5A-5H illustrate that blocking of VLA-3 improves survival. In FIGS. 5A and 5B, cells were isolated from the PL and lungs of septic mice (8 hours after CLP) injected with control peptide (88 μg/dose, IV) or LXY2 (88 μg/dose, IV). LXY2 is the cyclic peptide Cys_{D}-Asp_{D}-Gly-Tyr(3-NO₂)-Gly-4Hyp-Asn-Cys_{D} (Yao et al., “Discovery of Targeting Ligands for Breast Cancer Cells Using the One-Bead One-Compound Combinatorial Method,” J. Med. Chem. 52(1):126-133 (2009), the disclosure of which is incorporated herein by reference in its entirety). Anti-Ly6G (or anti-Gr-1) and anti-CD11b antibodies were used to stain neutrophils. The bar graphs show the total number of neutrophils in the PL and lungs (n=5). In FIG. 5C, the bar graphs show the total number of neutrophils in the PL of endotoxemia animals at 8 hours. In FIG. 5D, to determine the bacterial load in the PL of the three groups of mice, diluted samples were streaked on TSA blood agar and colonies were counted after 24 hours incubation at 37° C. (n=5). CLP surgery and endotoxemia was performed, and survival was analyzed using the Kaplan-Meyer log-rank test to compare controls (n=9) to LXY2 (n=9) treated mice. In FIG. 5E, serum levels of IL-6 were measured by sandwich ELISA. The graph shows the concentrations of IL-6. *p<0.05. In FIGS. 5F and 5G, the effect of LXY2 on neutrophil migration in vivo was assessed by multiphoton intravital microscopy (MP-IVM) in the fMLP-superfused cremaster vessels of LysM-GFP mice after IV injections of control peptide or LXY2 (88 μg). In FIG. 5H, Transmission Electron Microscopy imaging of fMLP superfused cremaster microvasculature of mice administered LXY2. Abbreviations: P=PMN, L=Lumen, Peri=Pericyte, Endo=Endothelium. The results in FIGS. 5A, 5B, and 5E are expressed as the mean±SEM. *p<0.05.

FIGS. 6A-6I show that conditional depletion of VLA-3 improves survival. In FIG. 6A, conditional depletions of VLA-3 (α₃-cKO) and αᵥ (αᵥ-cKO) were achieved by crossing mice expressing Cre in the elastase (Ela) promoter with mice with floxed integrin genes (see Methods). Pups from breeding Ela-Cre and α₃/αᵥ flox mice were genotyped for heterozygous gene expression and mice showing the presence of both flox and mutant cre were used for further breeding. Deletion of integrin genes was confirmed with the presence of mutant bands (Cre; 185 bp, α₃; 427 bp with P1/P3, αᵥ; 150 bp). In FIG. 6B, surface expression of α₃ and αᵥ in the Gr1^{high} CD11b^{high} neutrophils was measured in the cells isolated from BM and PL of LPS injected WT or cKO mice. In FIG. 6C, cells were isolated from the PL and lungs of septic cKO mice (6 hours after CLP) and anti-Ly6G/Gr1 and anti-CD11b antibodies were used to stain neutrophils. The bar graphs show the total number of neutrophils in the PL and lungs (n=6). In FIG. 6D, cells were isolated from the lungs of septic cKO animals (6 hours) and stained with Ly6G/Gr1 and CD11b antibodies for flow cytometry. The graph shows the frequencies of Ly6G^{high}CD11b^{high} cells in the lungs. In FIG. 6E, the bacterial load in the PL of cKO mice at 6 hours after CLP (n=6/group) is shown. In FIG. 6F, survival was analyzed using the Kaplan-Meyer log-rank test to compare cKOs and Ela-Cre control mice n=12/group. In FIG. 6G, serum levels of IL-6 as measured by ELISA are shown. The graph shows the concentrations of IL-6 (n=6/group). In FIGS. 6H and 6I, the effect of VLA-3 depletion on neutrophil migration in vivo was assessed by MP-IVM in the fMLP-superfused cremaster vessels. Ela-Cre mice were used as a control for α₃-cKO. For FIGS. 6D, 6E, and 6F, the results are expressed as the mean±SEM. *p<0.05.

### DETAILED DESCRIPTION

A first aspect described herein relates to a method of diagnosing sepsis or sepsis risk in a subject. For purposes of this and other aspects, the target “subject” encompasses an animal including any mammal, particularly a human. In the context of diagnosing sepsis or risk of septic infection in a subject, the target subject encompasses any subject that is at risk of contracting a septic infection. Particularly susceptible subjects include infants and juveniles, as well as immunocompromised juveniles and adults, and elderly adults. However, any infant, juvenile, adult, or elderly adult or immunocompromised individual at risk for septic infection can be diagnosed in accordance with the methods described herein.

According to one approach, the method includes detecting the presence of a subpopulation of neutrophils having an elevated integrin VLA-3 (CD49c/CD29) expression level in the subject, whereby the presence of the subpopulation of neutrophils indicates that the subject has sepsis or a risk of sepsis.

According to another approach, the method includes contacting a biological sample from a subject with a reagent that binds specifically to integrin VLA-3 in the biological sample; detecting the reagent bound to integrin VLA-3 in the biological sample, and determining the expression level of integrin VLA-3 in the biological sample wherein an elevated level of integrin VLA-3 in the biological sample, relative to a control level of integrin VLA-3, indicates that the subject has sepsis or is at risk of developing sepsis.

As used herein, the term “sample” is a biological sample obtained from a subject. The biological sample can be any sample that contains neutrophils. Those skilled in the art will recognize that plasma, whole blood, or a sub-fraction of whole blood, may be used. The biological sample may also be serum or bone marrow. These various biological samples may be obtained using standard procedures for the recovery of the particular sample.

For example, a blood or serum sample may be obtained by use of a standard blood draw, as disclosed in U.S. Pat. No. 4,263,922 to White, the disclosure of which is incorporated herein by reference in its entirety. Generally, in a standard blood draw, blood is drawn through a needle assembly and handle system into a collection tube. Subsequent to the blood draw, the needle assembly and the handle are removed from an end of the tube and a separate cap is fitted over each end of the tube to retain the blood sample in the tube for analysis. In the case of humans, a finger prick with a lancet or a blood draw via standard venipuncture is also a convenient method to obtain a body fluid sample. The drawn blood may be exposed immediately to an anticoagulant to preclude coagulation thereof. Known anticoagulants include without limitation heparin, EDTA, D-Phe-Pro-Arg chloromethyl ketone dihydrochloride (“PPACK”), and sodium citrate.

Bone marrow samples can be obtained according to standard procedures known in the art. For example, bone marrow samples can be obtained using needle aspiration or other known techniques. In certain instances, cells can be isolated from a bone marrow sample using a Ficoll-Hypaq density gradient. Other procedures for obtaining bone marrow samples include bone biopsy devices such as a hollow cannula or needle. Regardless of the tool used to acquire the sample, the bone marrow sample can then be prepared for subsequent analysis (e.g., fixation and labeling). Samples may be obtained in accordance with the methods described in U.S. Patent Publ. No. 2014/0038177 to Silva et al., the disclosure of which is incorporated herein by reference in their entirety.

According to another embodiment, the sample can be obtained by peritoneal lavage. Briefly, diagnostic peritoneal lavage (DPL) is the introduction of a perforated dialysis catheter into the peritoneal cavity to obtain fluid for laboratory analysis. DPL is practiced by at least three distinct techniques, which employ slightly different equipment and methods. In all techniques, a patient is prepared by placement of a bladder catheter and nasogastric tube for decompression of bladder and stomach. In one technique (i.e., the closed technique), a dialysis catheter is placed through a small skin incision using a sharp trocar. This technique has been modified to reduce risk to abdominal viscera. The modified technique uses a Seldinger wire, which is termed a “wire through needle” approach. A J-tipped spring wire is passed into the pelvis through an 18-gauge short beveled introducer needle. The needle is withdrawn and a multifenestrated peritoneal lavage catheter is advanced into the pelvis over the guide wire. The J-wire is removed, and established techniques then are used to sample abdominal fluids.

Inflammation can be classified as either acute or chronic. Acute inflammation is the initial response of the body to harmful stimuli and is achieved by the activation of immune cells followed by the release of various mediators and increased movement of leukocytes from the blood into the injured tissues. Prolonged inflammation, known as chronic inflammation, leads to a progressive shift in the type of cells which are present at the site of inflammation and is characterized by simultaneous destruction and healing of the tissue from the inflammatory process. Sepsis is a serious medical condition that is characterized by a whole-body inflammatory state (called a systemic inflammatory response syndrome or SIRS) and the presence of a known or suspected infection. The body may develop this inflammatory response to microbes in the blood, urine, lungs, skin, or other tissues. Invasion of bacteria to otherwise sterile sites like the peritoneal cavity leads to the immediate initiation of an inflammatory response. Integral to this response are oxygen radicals that are primarily generated to kill microbes, but can also damage host structures through the peroxidation of membrane phospholipids (Hampton et al., “Inside the Neutrophil Phagosome: Oxidants, Meyloperoxidase, and Bacterial Killing,” Blood 92:3007-3017 (1998); WO 2013/104798 to Meier, the disclosure of which are incorporated herein by reference in their entirety).

The diagnosis of sepsis or sepsis risk may be used for three major types of sepsis characterized by the type of infecting organism. Gram-negative sepsis is the most common and has a case fatality rate of about 35%. The majority of these infections are caused by Escherichia coli, Klebsiella pneumonia, and Pseudomonas aeruginosa. Gram-positive pathogens such as the Staphylococci and Streptococci are the second major cause of sepsis. The third major group includes the fungi, with fungal infections causing a relatively small percentage of sepsis cases, but with a high mortality rate. See WO 1999/064070 to Kink, the disclosure of which is incorporated herein by reference in its entirety.

In one embodiment, the subpopulation of neutrophils in mice is Gr1^{high}, CD11b^{high}. Gr1 is a murine protein also known as Ly-6G (Protein Database Accession No. P35461; see also Fleming et al., “Characterization of Two Novel Ly-6 genes. Protein Sequence and Potential Structural Similarity to Alpha-Bungarotoxin and Other Neurotoxins,” J. Immunol. 150 (12) 5379-5390 (1993), the disclosure of which are incorporated herein by reference in their entirety). A glycosylphosphatidylinositol (GPI)-linked protein, Gr1 is expressed by the myeloid lineage in a developmentally regulated manner in the bone marrow. While monocytes only express Gr1 transiently during their bone marrow development, the expression of Gr1 on bone marrow granulocytes as well as on peripheral neutrophils is a good marker for these populations.

The homologue or equivalent of Gr1 in non-murine cells can be identified using conventional molecular techniques. For example, the neutrophil subpopulation in humans may alternatively be the neutrophil-specific markers CD16+CD62L+. One skilled in the art can generate or obtain a cDNA or genomic DNA library prepared from myeloid cells from the organism of interest and use degenerate oligonucleotide primers designed using amino acid motifs or sequences from Gr1, for example to obtain a nucleic acid probe molecule to screen genomic or cDNA libraries; one might also use genetic approaches based on protein-protein interactions (e.g. a yeast two-hybrid system). A variety of publicly available bioinformatics resources may be used to screen sequence databases for homologues of murine Gr1. See U.S. Patent Publ. No. 2004/0229354 to Cambier et al., the disclosure of which is incorporated herein by reference in its entirety.

The neutrophil subpopulation may, alternatively, be defined by the neutrophil specific markers CD14+CD16-(Soehnlein et al., “Phagocyte Partnership During the Onset and Resolution of Inflammation,” Nat. Rev. Immunol. 10:427-439 (2010), the disclosure of which is incorporated herein by reference in its entirety). In a further embodiment, the neutrophil specific markers CD14+CD16+ may define the neutrophil subpopulation (Saha et al., “Multifunctional Monocytes: Toward A Functional Characterization of Blood Monocytes” Immunol. Cell. Biol. 89:2-4 (2011), the disclosure of which is incorporated herein by reference in its entirety).

The detecting step as recited in the several embodiments may be repeated at spaced intervals over a period of time. The determination of whether the subject has sepsis or is at risk of sepsis may be completed immediately following exposure to an infecting organism, or at any time thereafter. The determination may be used as a method to determine follow up treatment, by testing the selected subject's integrin VLA-3 expression levels or the presence of a subpopulation of neutrophils having elevated integrin VLA-3 levels at various time points during and after treatment for sepsis. Ideally, determination of sepsis or risk of sepsis is completed by obtaining body fluid samples soon after exposure to an infecting organism, e.g. within the first 24 hours after the exposure. The body fluid sample may be obtained from the subject more than 24 hours after the exposure, or within less time (e.g., within about six hours after exposure occurs). Additional body fluid samples may be further obtained within hours, days, or weeks after exposure to an infecting organism.

In accordance with one embodiment, the biomarker used to determine whether a subject has sepsis or is at risk for sepsis is integrin VLA-3. Integrin VLA-3 expression on neutrophils is strongly correlated with the severity of sepsis and can distinguish sepsis from SIRS. Currently, this is not possible within 2-3 days of diagnosis because this decision is made based on the detection of pathogens for sepsis, which takes more than 24 hours of bacteria culture in the lab. The methods described herein afford a significant improvement in the ability to discriminate between sepsis and SIRS using the biomarker integrin VLA-3. This can improve outcomes in both sepsis and SIRS cases, because therapeutic approaches to sepsis and SIRS are significantly different and appropriate therapeutic intervention can begin at a much earlier time point when using the diagnostic methods disclosed herein.

Detecting presence of integrin VLA-3 is carried out by contacting the biological sample from the subject with a reagent that binds specifically to integrin VLA-3 in the obtained biological sample. In one embodiment, the method further includes contacting the biological sample with a reagent that binds specifically to a neutrophil-specific marker.

The presence of a subpopulation of neutrophils having an elevated integrin VLA-3 expression level is found in individuals having sepsis or at risk of sepsis. As used herein, the term “elevated” is intended to mean that the measured integrin VLA-3 levels in the obtained biological sample are higher than either a predicted normal range for non-sepsis subjects, a threshold VLA-3 level, or a control sample (e.g., one or more calibration standards) measured simultaneously or previously. A control sample can be from the same subject at a time prior to exposure to a sepsis infecting organism, from a different subject or panel of subjects not exposed to a sepsis infecting organism, or one or more calibration samples each containing a known quantity of integrin VLA-3. The presence of increased integrin VLA-3 levels compared to control or normal range integrin VLA-3 levels are identified as a means to detect sepsis or risk of sepsis.

The concentration of the VLA-3 biomarker may be measured by using standard immunodiagnostic techniques, including immunoassays such as competition, direct reaction, or sandwich type assays. Such assays include, but are not limited to, Western blots, agglutination tests, enzyme-labeled and mediated immunoassays such as ELISAs, biotin/avidin type assays, radioimmunoassay, immunoelectrophoresis, immunoprecipitation, gas chromatography, high performance liquid chromatography (HPLC), size exclusion chromatography, solid-phase affinity, flow cytometry, etc. In one particular embodiment, the detecting is carried out using flow cytometry. The concentration of the VLA-3 biomarker may also be measured by any type applied in the field of diagnostics, including but not restricted to assay methods based on enzymatic reactions, luminescence, in particular fluorescence or radio chemicals. The detection methods comprise rapid test formats including immunochromatography (e.g., strip formats), radioimmunoassays, chemiluminescence- and fluorescence-immunoassays, immunoblot assays, enzyme-linked immunoassays (ELISA), luminex-based bead arrays, and protein microarray assays. See U.S. Pat. Publ. No. 2011/0086831 to Bergmann et al., the disclosure of which is incorporated herein by reference in its entirety. The assay types can further be microtitre plate-based, chip-based, bead-based, wherein the biomarker proteins can be attached to the surface or in solution. The assays can be homogenous or heterogeneous assays, sandwich assays, competitive and non-competitive assays (THE IMMUNOASSAY HANDBOOK, Ed. David Wild, Elsevier LTD, Oxford; 3rd ed. (May 2005); Hultschig et al., “Recent Advances of Protein Microarrays,” Curr. Opin. Chem. Biol. 10(1):4-10 (2006), the disclosure of which are incorporated herein by reference in their entirety).

Fluorescence based assays as used herein comprise the use of dyes that label the reagent. Exemplary dyes, may for instance be selected from the group comprising FAM (5- or 6-carboxyfluorescein), VIC, NED, Fluorescein, Fluorescein-isothiocyanate (FITC), IRD-700/800, Cyanine dyes such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethoxy-fluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides such as Hoechst 33258; Phenanthridines such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like. Chemiluminescence based assays comprise the use of dyes, based on the physical principles described for chemiluminescent materials in KIRK-OTHMER, ENCYCLOPEDIA OF CHEMICAL TECHNOLOGY, 4th ed., exec. Ed., J. I. Kroschwitz; Editor, M. Howe-Grant, John Wiley & Sons, 15:518-562 (1993), the disclosure of which is incorporated herein by reference in its entirety.

Lateral flow tests based on the principles of chromatographic immunoassay may also be used in this aspect. Lateral flow tests use strips coated with antibodies and/or other reactants that, upon reaction with VLA-3 present in a contacting blood specimen or other sample, result in the appearance of colored lines (i.e., an indicator) indicative of the presence in a patient of integrin VLA-3 above a certain threshold value, which triggered the formation of VLA-3/antibody complexes. The general format of the strip exhibits a single test line, so-called “T-line” and a single control line, so-called “C-line”. The control line is used as an indicator of functional validity. More recent test strips are offered that group multiple test lines for the detection of more than one kind of substance in the contacted sample. See U.S. Pat. Publ. No. US2004/0191760 to Zhou et al., the disclosure of which is incorporated herein by reference in its entirety.

Reagents that bind to integrin VLA-3 include, without limitation, any known protein or peptide such as an antibody, antibody binding fragment, or antibody mimic, nucleic acid aptamer, and small molecule integrin VLA-3 reagents.

In one embodiment of the present methods, the reagent is a monoclonal antibody, or binding fragment thereof, aptamer, or antibody mimic. As used herein, the term “antibody” may exist in a variety of forms including, for example, polyclonal antibodies, monoclonal antibodies, intracellular antibodies (“intrabodies”), antibody fragments (e.g. Fv, Fab and F(ab)2), as well as single chain antibodies (scFv), chimeric antibodies, and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. U.S.A. 85:5879-5883 (1988); Bird et al, “Single-Chain Antigen-Binding Proteins,” Science 242:423-426 (1988), the disclosure of which are incorporated herein by reference in their entirety).

Naturally occurring antibodies typically have two identical heavy chains and two identical light chains, with each light chain covalently linked to a heavy chain by an inter-chain disulfide bond and multiple disulfide bonds further link the two heavy chains to one another. Individual chains can fold into domains having similar sizes (110-125 amino acids) and structures, but different functions. The light chain can comprise one variable domain (VL) and/or one constant domain (CL). The heavy chain can also comprise one variable domain (VH) and/or, depending on the class or isotype of antibody, three or four constant domains (CH1, CH2, CH3 and CH4). In humans, the isotypes are IgA, IgD, IgE, IgG, and IgM, with IgA and IgG further subdivided into subclasses or subtypes (IgA1-2 and IgG1-4).

Generally, the variable domains show considerable amino acid sequence variability from one antibody to the next, particularly at the location of the antigen-binding site. Three regions, called hyper-variable or complementarity-determining regions (CDRs), are found in each of VL and VH, which are supported by less variable regions called framework variable regions. The inventive antibodies include IgG monoclonal antibodies as well as antibody fragments or engineered forms. These are, for example, Fv fragments, or proteins wherein the CDRs and/or variable domains of the exemplified antibodies are engineered as single-chain antigen-binding proteins.

The portion of an antibody made up of the VL and VH domains is designated as an Fv (Fragment variable) and constitutes the antigen-binding site. A single chain Fv (scFv or SCA) is an antibody fragment containing a VL domain and a VH domain on one polypeptide chain, wherein the N terminus of one domain and the C terminus of the other domain are joined by a flexible linker. The peptide linkers used to produce the single chain antibodies are typically flexible peptides, selected to assure that the proper three-dimensional folding of the VL and VH domains occurs. The linker is generally 3 to 50 amino acid residues, and in some cases is shorter, e.g., about 3 to 30 amino acid residues, or 3 to 25 amino acid residues, or even 3 to 15 amino acid residues. An example of such linker peptides includes repeats of four glycine residues followed by a serine residue.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Methods for monoclonal antibody production are well-known in the art (MONOCLONAL ANTIBODIES—PRODUCTION, ENGINEERING AND CLINICAL APPLICATIONS (Mary A. Ritter and Heather M. Ladyman eds., 1995), the disclosure of which is incorporated herein by reference in its entirety). Monoclonal antibodies can be prepared using hybridoma methods, such as those described by Kohler and Milstein, “Continuous Cultures of Fused Cells Secreting Antibody of Predefined Specificity,” Nature 256:495-497 (1975), the disclosure of which is incorporated herein by reference in its entirety. Using the hybridoma method, a host animal is immunized to elicit the production by lymphocytes of antibodies that will specifically bind to an immunizing antigen. Generally, the process involves obtaining immune cells (lymphocytes) from the spleen of a mammal which has been previously immunized with the antigen of interest. In one embodiment, monoclonal antibodies are raised against the VLA-3 peptide. The antibody-secreting lymphocytes are then fused with myeloma cells or transformed cells, thereby producing an immortal, immunoglobulin-secreting cell line (Milstein and Kohler, “Derivation of Specific Antibody-Producing Tissue Culture and Tumor Lines by Cell Fusion,” Eur. J. Immunol. 6:511 (1976), the disclosure of which is incorporated herein by reference in its entirety). The resulting fused cells, or hybridomas, are cultured, and screened for the production of the desired monoclonal antibodies. Colonies producing such antibodies are cloned, and grown either in vivo or in vitro to produce large quantities of antibody. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against integrin VLA-3, as determined by immunoprecipitation, immunoblotting, or by an in vitro binding assay such as radioimmunoassay (RIA) or enzyme-linked immunosorbent assay (ELISA) can then be propagated either in in vitro culture using standard methods (JAMES W. GODING, MONOCLONAL ANTIBODIES: PRINCIPLES AND PRACTICE (Academic Press 1986), the disclosure of which is incorporated herein by reference in its entirety) or in vivo as ascites tumors in an animal. The monoclonal antibodies can then be purified from the culture medium or ascites fluid as described for polyclonal antibodies above.

In another embodiment, antibodies or antibody fragments can be isolated from antibody phage libraries generated using the techniques described in McCafferty et al., “Phage Antibodies: Filamentous Phage Displaying Antibody Variable Domains,” Nature 348:552-554 (1990), the disclosure of which is incorporated herein by reference in its entirety. Li et al., “Platelet Fragmentation Requires a Specific Structural Conformation of Human Monoclonal Antibody Against β3 Integrin,” J. Biol. Chem. 283(6):3224-3230 (2008 the disclosure of which is incorporated herein by reference in its entirety, describes the isolation of nine monoclonal human antibodies capable of binding to VLA-3 from a human scFv antibody library using phage surface display technology. Thus, this technology is a very viable alternative to traditional monoclonal antibody hybridoma techniques for isolation of monoclonal antibodies.

Alternatively monoclonal antibodies can also be made using recombinant DNA methods as described in U.S. Pat. No. 4,816,567 to Cabilly et al., the disclosure of which is incorporated herein by reference in its entirety. Polynucleotides encoding a monoclonal antibody are isolated from mature B-cells or hybridoma cells by RT-PCR using oligonucleotide primers that specifically amplify the genes encoding the heavy and light chains of the antibody. The isolated polynucleotides encoding the heavy and light chains are then cloned into suitable expression vectors, which when transfected into host cells, monoclonal antibodies are generated by the host cells. Also, recombinant monoclonal antibodies or fragments thereof of the desired species can be isolated from phage display libraries as described (McCafferty et al., “Phage Antibodies: Filamentous Phage Displaying Antibody Variable Domains,” Nature 348:552-554 (1990); Clackson et al., “Making Antibody Fragments Using Phage Display Libraries,” Nature 352:624-628 (1991); and Marks et al., “By-passing Immunization: Human Antibodies from V-gene Libraries Displayed on Phage,” J. Mol. Biol. 222:581-597 (1991), the disclosure of which are incorporated herein by reference in their entirety).

The monoclonal antibody can be a humanized antibody. Such antibodies are used therapeutically to reduce antigenicity and human anti-mouse antibody responses when administered to a human subject. A human antibody is an antibody produced by a human or an antibody having an amino acid sequence corresponding to an antibody produced by a human. An antibody can be humanized by substituting the complementarity determining region (CDR) of a human antibody with that of a non-human antibody (e.g., mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), the disclosure of which are incorporated herein by reference in their entirety). Humanized antibodies can be produced using various techniques known in the art. Immortalized human B lymphocytes immunized in vitro or isolated from an immunized individual that produce an antibody directed against a target antigen can be generated (See e.g., Reisfeld et al., MONOCLONAL ANTIBODIES AND CANCER THERAPy 77 (Alan R. Liss ed., 1985) and U.S. Pat. No. 5,750,373 to Garrard, the disclosure of which are incorporated herein by reference in their entirety). Also, the humanized antibody can be selected from a phage library, where that phage library expresses human antibodies (Li et al., “Platelet Fragmentation Requires a Specific Structural Conformation of Human Monoclonal Antibody Against P3 Integrin,” J. Biol. Chem. 283(6):3224-3230 (2008), Vaughan et al., “Human Antibodies with Sub-Nanomolar Affinities Isolated from a Large Non-immunized Phage Display Library,” Nature Biotechnology 14:309-314 (1996); Sheets et al., “Efficient Construction of a Large Nonimmune Phage Antibody Library: The Production of High-Affinity Human Single-Chain Antibodies to Protein Antigens,” Proc. Nat'l. Acad. Sci. U.S.A. 95:6157-6162 (1998); Hoogenboom et al., “By-passing Immunisation. Human Antibodies From Synthetic Repertoires of Germline VH Gene Segments Rearranged In Vitro,” J. Mol. Biol. 227:381-8 (1992); Marks et al., “By-passing Immunization. Human Antibodies from V-gene Libraries Displayed on Phage,” J. Mol. Biol. 222:581-97 (1991), the disclosure of which are incorporated herein by reference in their entirety). Humanized antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., the disclosure of which are incorporated herein by reference in their entirety.

In addition to utilizing whole antibodies, the methods herein encompass use of binding portions of such antibodies. Such binding portions include Fab fragments, F(ab)₂ fragments, Fab′ fragments, F(ab′)₂ fragments, Fd fragments, Fd′ fragments, Fv fragments, and minibodies, e.g., 61-residue subdomains of the antibody heavy-chain variable domain (Pessi et al., “A Designed Metal-binding Protein with a Novel Fold,” Nature 362:367-369 (1993), the disclosure of which is incorporated herein by reference in its entirety). Domain antibodies (dAbs) (see, e.g., Holt et al., “Domain Antibodies: Proteins for Therapy,” Trends Biotechnol. 21:484-90 (2003), the disclosure of which is incorporated herein by reference in its entirety) are also suitable for the methods disclosed herein. These antibody fragments can be made by conventional procedures, such as proteolytic fragmentation procedures, as described in J. Goding, MONOCLONAL ANTIBODIES: PRINCIPLES AND PRACTICE 98-118 (1984), the disclosure of which is incorporated herein by reference in its entirety.

Further, single chain antibodies are also suitable (e.g., U.S. Pat. No. 5,476,786 to Huston and U.S. Pat. No. 5,132,405 to Huston & Oppermann; Huston et al., “Protein Engineering of Antibody Binding Sites: Recovery of Specific Activity in an Anti-digoxin Single-chain Fv Analogue Produced in Escherichia coli,” Proc. Nat'l Acad. Sci. USA 85:5879-83 (1988); U.S. Pat. No. 4,946,778 to Ladner et al.; Bird et al., “Single-chain Antigen-binding Proteins,” Science 242:423-6 (1988); Ward et al., “Binding Activities of a Repertoire of Single Immunoglobulin Variable Domains Secreted from Escherichia coli,” Nature 341:544-6 (1989), each of which the disclosure is incorporated herein by reference in its entirety). Single chain antibodies are formed by linking the heavy and light immunoglobulin chain fragments of the Fv region via an amino acid bridge, resulting in a single chain polypeptide. The use of univalent antibodies is also embraced herein. sdAbs can be naturally produced, i.e., by immunization of dromedaries, camels, llamas, alpacas or sharks (Ghahroudi et al., “Selection and Identification of Single Domain Antibody Fragments from Camel Heavy-Chain Antibodies,” FEBS Letters 414(3): 521-526 (1997), the disclosure of which is incorporated herein by reference in its entirety). Alternatively, the antibody can be produced in microorganisms or derived from conventional whole antibodies (Harmsen et al., “Properties, Production, and Applications of Camelid Single-Domain Antibody Fragments,” Appl. Microbiol. Biotechnology 77:13-22 (2007), Holt et al., “Domain Antibodies: Proteins for Therapy,” Trends Biotech. 21(11): 484-490 (2003), the disclosure of which are incorporated herein by reference in their entirety).

Tribodies are multifunctional recombinant antibody derivatives that combine two scFv fragments with a Fab fragment. The Fab fragment serves as a specific heterodimerization signal, and the two scFv fragments are each fused to a different Fab chain (Schoonjans et al., “Efficient Heterodimerization of Recombinant Bi- and Trispecific Antibodies,” Bioseparation 9(3): 179-183 (2000), the disclosure of which is incorporated herein by reference in its entirety).

Diabodies are small bivalent and bispecific antibody fragments that comprise a heavy (VH) chain variable domain connected to a light chain variable domain (VL) on the same polypeptide chain (VH-VL) connected by a peptide linker that is too short to allow pairing between the two domains on the same chain (Hollinger et al., “Diabodies”: Small Bivalent and Bispecific Antibody Fragments,” Proc. Natl. Acad. Sci. U.S.A. 90(14):6444-6448 (1993), the disclosure of which is incorporated herein by reference in its entirety).

Exemplary antibodies against VLA-3 include, without limitation, those commercially available as LS-A8136 (Lifespan Biosciences), LS-A8140 (Lifespan Biosciences), LS-C123048 (Lifespan Biosciences), LS-C44108 (Lifespan Biosciences), PA1621 (Booster Immunoleader), MCA1948PE (AbD Serotec), MCA1813 (AbD Serotec), MCA1948FT (AbD Serotec), MCA1948 (AbD Serotec), HPA008572 (Atlas Antibodies), 17-0494-41 (eBioscience), 17-0494-42 (eBioscience), 343805 (BioLegend), 343804 (BioLegend), 343802 (BioLegend), 343801 (BioLegend), ABIN968338 (antibodies-online), ABIN112369 (antibodies-online), ABIN112368 (antibodies-online), ABIN112371 (antibodies-online), MAB1952Z (Merck Millipore), CP11L-100UG (Merck Millipore), AB1920 (Merck Millipore), MAP1952 (Merck Millipore), GWB-252CD8 (Genway), GWB-33C613 (Genway), GWB-390721 (Genway), GWB-4A5FF0 (Genway), PA1-28601 (Thermo Fisher Scientific, Inc.), MA1-27165 (Thermo Fisher Scientific, Inc.), MA1-34075 (Thermo Fisher Scientific, Inc.), MA1-70075 (Thermo Fisher Scientific, Inc.), 251177 (Abbiotec), 251428 (Abbiotec), orb14075 (Biorbyt), orb13512 (Biorbyt), AP20567PU-N (Acris Antibodies), BM6022P (Acris Antibodies), SM1544A (Acris Antibodies), BM6023P (Acris Antibodies), 69475 (NovaTeinBio), 119-17485 (Raybiotech), 119-17484 (Raybiotech), OASA02153 (Aviva Systems Biology), OASA02147 (Aviva Systems Biology), OASA02150 (Aviva Systems Biology), OASA02155 (Aviva Systems Biology), CABT-20410MH (Creative Biomart), CABT-29879MH (Creative Biomart), CABT-29877MH (Creative Biomart), CABT-29881MH (Creative Miomart), XW-7793 (ProSci), NBP1-40612 (Novus Biologicals), NBP1-61615 (Novus Biologicals), NB100-2675 (Novus Biologicals), NB100-64901 (Novus Biologicals), I7661-33D (United States Biological), I7661-33C (United States Biological), I7661-33C (United States Biological), 1761133D1 (United States Biological), I7661-33M (United States Biological), AF2787 (R&D Systems), MAB1345 (R&D Systems), FAB1345A (R&D Systems), FAB1345A (R&D Systems), FAB1345F (R&D Systems), sc-59967 (Santa Cruz Biotechnology), sc-32237 (Santa Cruz Biotechnology), sc-6587 (Santa Cruz Biotechnology), sc-6592 (Santa Cruz Biotechnology), bs-1057R (Bioss), bs-2093R (Bioss), ab131055 (abcam), ab133198 (abcam), ab11767 (abcam), ab125120 (abcam), ab140919 (abcam), ab20141 (abcam), ab91043 (abcam), ab20140 (abcam), ab115912 (abcam), ab91050 (abcam), ab30488 (abcam), ab30489 (abcam), ab181432 (abcam), and ab190731 (abcam).

Aptamers are a class of molecule that represents an alternative to antibodies in term of molecular recognition. Aptamers are peptides or oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk and Gold, “Systematic Evolution of Ligands by Exponential Enrichment: RNA Ligands to Bacteriophage T4 DNA Polymerase,” Science 249(4968):505-510 (1990), the disclosure of which is incorporated herein by reference in its entirety. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena, “Aptamers: An Emerging Class of Molecules that Rival Antibodies in Diagnostics,” Clin. Chem. 45(9):1628-1650 (1999), the disclosure of which is incorporated herein by reference in its entirety.

Antibody mimics are also suitable for use herein. Exemplary antibody mimics include, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Nat'l Acad. Sci. USA 99:1253-1258 (2002), each of which the disclosure is incorporated herein by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), the disclosure of which is incorporated herein by reference in its entirety). Variations in these antibody mimics can be created by substituting one or more domains of these polypeptides and then screening the modified monobodies or affibodies for integrin VLA-3 binding specificity.

Procedures for raising polyclonal antibodies are also well known. Polyclonal antibodies and fragments thereof can be raised according to known methods by administering the appropriate antigen or epitope to a host animal selected, e.g., from pigs, cows, horses, rabbits, goats, sheep, and mice, among others, and then recovering serum (containing the antibodies) from the host animal. Various adjuvants known in the art can be used to enhance antibody production. Procedures for raising polyclonal antibodies are disclosed in Ed Harlow and David Lane, USING ANTIBODIES: A LABORATORY MANUAL (Cold Spring Harbor Laboratory Press, 1988), the disclosure of which is incorporated herein by reference in its entirety. Although antibodies can be polyclonal, using purification techniques polyclonal antiserum can be rendered monospecific.

In particular embodiments, these detection methods involve contacting the obtained sample with a binding partner capable of selectively interacting with VLA-3 biomarker. In one embodiment, the binding partner is an anti-VLA-3 antibody, which refers to an antibody or a binding fragment thereof which recognizes integrin VLA-3. The anti-VLA-3 antibody can be polyclonal or monoclonal, ideally monoclonal, as well as any fragments thereof that bind specifically to integrin VLA-3. In another embodiment, the binding partner is an antibody mimic, which can be a nucleic acid or peptide aptamer, or a polypeptide scaffold containing one or more variable regions that bind specifically to integrin VLA-3.

The aforementioned binding assays may involve the binding of the binding partner (i.e. antibody, antibody mimic, or aptamer) to a solid support. Solid supports which can be used include, without limitation, substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

The binding partners to be used in these assays may be labeled with a detectable molecule or substance, such as a fluorescent molecule of the type described above, a radioactive molecule or any others labels known in the art. Labels are known in the art that generally provide (either directly or indirectly) a signal. As used herein, the term “labeled”, with regard to the binding partner, is intended to encompass direct labeling of the binding partner by coupling (i.e., physically linking) a detectable substance, such as a radioactive agent or a fluorophore (e.g., fluorescein isothiocyanate (FITC) or phycoerythrin (PE) or Indocyanine (Cy5)) to the binding partner, as well as indirect labeling of the binding partner by reactivity with a detectable substance. A binding partner may be labeled with a radioactive molecule by any method known in the art. For example radioactive molecules include, without limitation, I¹²³, I¹²⁴, In¹¹¹, Re¹⁸⁶, and Re¹⁸⁸. Depending upon the assay and detection methods used, antibodies may optionally be conjugated to other proteins or chemical markers to facilitate detection of the antibody binding to VLA-3. Antibodies may be conjugated to enzymatic elements such as alkaline phosphatase, or readily detectable groups such as colloidal gold, biotin, and streptavidin. Other suitable conjugation agents are well known in the art. See U.S. Pat. Publ. No. 2004/0115748 to Kelley, the disclosure of which is incorporated herein by reference in its entirety.

VLA-3 and neutrophil markers may be detected by flow cytometry. Flow cytometry has been widely used to identify leukocyte populations in the central nervous system of experimental murine models of chronic demyelinating diseases as well in a murine model of cerebral ischemia (see Tjoa et al., “The Use of Flow Cytometry to Assess Neutrophil Infiltration in the Injured Murine Spinal Cord,” J. Neurosci. Meth. 129:49-59 (2003), the disclosure of which is incorporated herein by reference in its entirety). Flow cytometry can measure integrin expression on neutrophils recovered in a particular sample. RBC lysis may be performed using ACK lysing buffer (Invitrogen, San Diego, Calif.). Samples, with or without prior dilution, may be stained with, e.g., labeled anti-Gr1, labeled anti-Ly6G, and labeled anti-CD11b (eBioscience, San Diego, Calif.,), among others. The samples may be fixed with any suitable fixative such as, e.g., ultracold alcohol or 3.7% formaldehyde. Cell data can be collected on FACS Calliber flow cytometer (BD Biosciences, San Diego, Calif.) or an equivalent flow cytometer. At the indicated time points a sample is removed, and the forward and side scatter parameters of the cells as well as cell surface marker fluorescence are measured by flow cytometry using, for example, a Becton Dickenson FACScan equipped with CellQuest software.

In another embodiment, an ELISA method is used for measuring the integrin VLA-3 concentration, wherein the wells of a microtiter plate are coated with a set of antibodies that bind specifically to integrin VLA-3 or neutrophil-specific markers, such as Gr-1, CD14, CD16, or CD62. The body fluid sample, either with or without prior dilution, is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured integrin VLA-3, the plate washed, and the presence of the secondary binding molecule detected using methods well known in the art.

In another embodiment, the integrin VLA-3 concentration of the body fluid sample may be performed using an array chip. Such an array technology allows a large number of experiments to be performed simultaneously on a single substrate, commonly known as a biochip when used for biological analytes. For example the binding partner for integrin VLA-3 or neutrophil-specific markers, such as Gr-1, CD14, CD16, or CD62 may be immobilized at the surface of the array chip, either with or without prior dilution. The body fluid sample obtained from the subject is optionally diluted and then deposited on the array chip. After a period of incubation sufficient to allow the formation of binding partner-VLA-3 complexes, the array chip is then washed to remove unbound moieties, and thus allowing the isolation of VLA-3 positive neutrophils or neutrophils generally. In a second step, the measurement of integrin VLA-3 concentration may be performed with a suitable detection protocol for label-free detection, or using a suitable detection protocol for labeled detection of a second binding partner specific for integrin VLA-3. In one embodiment, the second binding partner is labeled, thus allowing the formation of a set of “spots” (colored deposit) specific for integrin VLA-3. For example, detection and quantification may be performed by analyzing the spots on the array chip with a specific detector.

The above-identified capture assays can also be performed after isolation of neutrophil populations generally, or VLA-3+ neutrophil populations by FACS sorting, and then disrupting cells to liberate free surface markers for protein quantitation.

The determination of sepsis or risk of sepsis can be made based on the presence of or level of the VLA-3 biomarker, or it can be based on additional diagnostic markers or biomarkers. There are a number of additional biomarkers that can be used to assist in detecting or diagnosing sepsis or risk of sepsis. These biomarkers include, but are not limited to, activated partial thromboplastin time (aPTT), CD11b, CD25, CD64, complement peptides (C3, C4, C5a), elastase alpha 1 proteinase inhibitor complex, endothelial-leukocyte adhesion molecule 1 (ELAm-1), endocan, E-Selectin, fibrin degradation products, growth-arrest-specific protein 6 (gas-6), granulocyte colony-stimulating factor (G-CSF), gelsolin, IL-1 receptor antagonists, IL-8, IL-10, IL-12, IL-18, Interferon-induced protein 10 (IP-10), laminin, Lipopolysaccharide binding protein (LBP), nitric oxide (NO), nitrate, nitrite, osteopontin, plasminogen activator inhibitor 1 (PAI-1), pentraxin 3, peptidoglycan, plasma fibronectin (pFN), Group II phospholipase A2 (PLA2-II), serum lysozymes, soluble ST2 protein, surfactant proteins (A, B, C, D), Triggering receptor expressed on myeloid cells 1 protein (TREM-1), and troponin. Biomarkers for sepsis are further discussed in Pierrakos et al., “Sepsis Biomarkers: A Review,” Critical Care 14:R15 (2010), the disclosure of which is incorporated herein by reference in its entirety.

The subject may be an individual who previously had sepsis and said detecting is used to determine risk of reoccurrence of sepsis in the subject. In one particular embodiment, the subject is an individual with a predisposition to sepsis and said method is used for early detection of the sepsis.

After a subject is evaluated using the disclosed methods to assess the presence of sepsis or risk of sepsis, the result of the diagnosis will determine the course of treatment, if any, for the tested subject.

Thus, another aspect relates to a method of treating a patient for sepsis. The method includes performing one of the methods disclosed herein to diagnose the patient with sepsis and administering a therapy to treat the patient for sepsis.

In one embodiment, when the patient has sepsis and/or an increased sepsis risk based on said diagnosing, the therapy for the subject comprises administration of VLA-3 antagonist peptides or anti-VLA-3 antibody. Exemplary VLA-3 antagonist peptides include, without limitation, the cyclic peptides LXY1 and LXY2 (Cys_{D}-Asp_{D}-Gly-Leu-Gly-4Hyp-Asn-Cys_{D} and Cys_{D}-Asp_{D}-Gly-Tyr(3-NO₂)-Gly-4Hyp-Asn-Cys_{D}, respectively) (Yao et al., “Discovery of Targeting Ligands for Breast Cancer Cells Using the One-Bead One-Compound Combinatorial Method,” J. Med. Chem. 52(1):126-133 (2009), the disclosure of which is incorporated herein by reference in its entirety), Accutin (Yeh et al., “Accutin, A New Disintegrin, Inhibits Angiogenesis In Vitro and In Vivo by Acting as Integrin Alphavbeta3 Antagonist and Inducing Apoptosis,” Blood 1998 92(9):3268-76 (1998), the disclosure of which is incorporated herein by reference in its entirety), and Gly-Arg-Gly-Asp (GRGD) (Chen et al., “Signaling and Regulatory Mechanisms of Integrinalpha3beta1 on the Apoptosis of Cultured Rat Podocytes,” J. Lab Clin. Med. 147(6):274-80 (2006), the disclosure of which is incorporated herein by reference in its entirety). Exemplary anti-VLA-3 antibodies include those described above or humanized variants thereof.

In another embodiment, when the patient has sepsis and/or an increased sepsis risk based on said diagnosing, the therapy for the patient comprises depletion of integrin VLA-3 expression. In yet another embodiment, when the patient has sepsis and/or an increased sepsis risk based on said diagnosing, the therapy for the patient comprises a drug, therapy, surgery, or any combination thereof.

The method of treating may also include a drug that is an agent for managing sepsis resistance, a blocker of VLA-3, an antibiotic, a vasopressor, or a combination thereof These agents may be administered in a single or in the form of multiple distinct compositions.

VLA-3 blockers include, without limitation, antisense molecules, siRNA molecules, shRNA molecules, and miRNA molecules.

Antisense nucleic acid molecules capable of hybridizing with an RNA transcript coding for integrin VLA-3 are expressed from a transgene which is prepared by ligation of a DNA molecule, coding for integrin VLA-3, or a fragment or variant thereof, into an expression vector in reverse orientation with respect to its promoter and 3′ regulatory sequences. Upon transcription of the DNA molecule, the resulting RNA molecule will be complementary to the mRNA transcript coding for the actual protein or polypeptide product. Ligation of DNA molecules in reverse orientation can be performed according to known techniques which are standard in the art. Recombinant molecules including an antisense sequence or oligonucleotide fragment thereof, may be directly introduced into cells of tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes.

As an alternative to full-length antisense VLA-3 mRNA, siRNA can be used to bind to VLA-3. siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the VLA-3 nucleotide sequence. siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule.

Various improvements of siRNA compositions, such as the incorporation of modified nucleosides or motifs into one or both strands of the siRNA molecule to enhance stability, specificity, and efficacy, have been described and are suitable for use in accordance with this aspect (see e.g., WO 2004/015107 to Giese et al.; WO 2003/070918 to McSwiggen et al.; WO 1998/39352 to Imanishi et al.; U.S. Patent Application Publ. No. 2002/0068708 to Jesper et al.; U.S. Patent Application Publ. No. 2002/0147332 to Kaneko et al; U.S. Patent Application Publ. No. 2008/0119427 to Bhat et al., the disclosure of which are incorporated herein by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway. shRNA molecules that effectively interfere with human integrin VLA-3 expression have been developed and are suitable for use in the methods described herein.

The additional therapies for use in any aspect described herein include, without limitation, oxygen administration, fluid administration, dialysis, surgery that removes sources of septic infection including pus and abscesses, or any of the aforementioned pharmaceutical compositions. Exemplary RNAi against VLA-3 include, without limitation, H00003675-R01 (Novus Biologicals) and H00003675-R02 (Novus Biologicals). Methods for selecting an appropriate RNA or RNA-encoding vectors are well known in the art for genes whose sequence is known (e.g. see Tuschl et al., “Targeted mRNA Degradation by Double-Stranded RNA In Vitro,” Genes Dev. 13:3191-7 (1999); Hannon, G J., “RNA Interference,” Nature 418:244-251 (2002); McManus et al., “Gene Silencing Using Micro-RNA Designed Hairpins,” RNA 8:842-850 (2002); U.S. Pat. No. 6,573,099 to Graham, U.S. Pat. No. 6,506,559 to Fire et al., WO 01/36646 to Zernicka-Goetz et al., WO 99/32619 to Fire et al., and WO 01/68836 to Beach et al., the disclosure of which are incorporated herein by reference in their entirety.

Pharmaceutical compositions for treating a patient with sepsis also contain pharmaceutically or physiologically acceptable carriers, excipients, or stabilizers. The pharmaceutical compositions can be in solid or liquid form such as, tablets, capsules, powders, solutions, suspensions, or emulsions, and can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by introduction into one or more lymph nodes. For most therapeutic purposes, peptides or nucleic acids can be administered intravenously or parenterally.

For injectable dosages, solutions or suspensions of the one or more therapeutic agents can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are liquid carriers, particularly for injectable solutions.

For use as aerosols, the one or more therapeutic agents in solution or suspension may be packaged in a pressurized aerosol container together with suitable propellants, for example, hydrocarbon propellants like propane, butane, or isobutane with conventional adjuvants. The materials also may be administered in a non-pressurized form such as in a nebulizer or atomizer.

Other delivery systems that are known in the art can be modified for delivery of the therapeutic proteins, peptides, or nucleic acid molecules described supra.

A further aspect involves monitoring the efficacy of any sepsis therapy. In one embodiment, this is carried out by determining whether a baseline integrin VLA-3 level is greater than the post-therapy integrin VLA-3 level. If so, then the therapy is effective. Alternatively, if the baseline integrin VLA-3 level is less than the post-therapy integrin VLA-3 level, then the therapy is not effective and a new therapeutic intervention should be selected.

A suitable reference level of integrin VLA-3 expression measures sepsis or sepsis risk in a sample obtained from a non-disease tissue, but not necessarily, derived from the same subject that is being diagnosed. In one embodiment, the reference level of integrin VLA-3 expression is obtained from a sample representing the same tissue or cell type as the sample being used for the diagnosis. The reference level may be integrin VLA-3 expression in a normal, non-diseased, sample from the subject. As an alternative, a reference level can be obtained from a non-diseased tissue from a population of individuals.

The method of the present aspect described herein may further include repeating said administering in spaced intervals over a period of time. In one embodiment, the therapy is carried out in spaced intervals over a period of time, wherein the baseline integrin VLA-3 level is obtained at an intermediate point within the period of time and the post-therapy integrin VLA-3 level is obtained after that immediate point.

A fourth aspect relates to a method of discriminating between sepsis and systemic inflammatory response syndrome (SIRS). The method includes detecting the presence or absence of a subpopulation of neutrophils having an elevated integrin VLA-3 expression level in a subject having systemic inflammation, whereby the presence of the subpopulation of neutrophils indicates that the subject has sepsis and the absence of the subpopulation of neutrophils indicates that that subject has SIRS. This method is carried out in accordance with the aspects described supra.

In one embodiment, the method further includes selecting a patient exhibiting one or more clinical symptoms of sepsis and/or SIRS prior to said detecting. After discriminating between the two conditions, the appropriate therapies can be administered. Sepsis treatments are described above.

The therapy of the present aspect described herein may include, but is not limited to, agents selected from the group made up of TNF-a and IL-1 receptor antagonists, antibradykinin, platelet-activating factor receptor antagonists, anticoagulants (antithrombin III), bradykinin antagonist, deltibant (CP-0127), epinephrine, steroids, and diphenhydramine. Alternatively, the therapy may include an antioxidant selected from the group made up of selenium, glutamine, eicosapentaenoic acid, melatonin, vitamin C, and vitamin E.

For further discussion of treatment of SIRS, see Fein et al., “Treatment of Severe Systemic Inflammatory Response Syndrome and Sepsis With a Novel Bradykinin Antagonist, Deltibant (CP-0127). Results of Randomized, Double-Blind, Placebo-Controlled Trial. CP-0127 SIRS and Sepsis Study Group,” JAMA 277(6):482-87 (1997); Berger et al., “Antioxidant Supplementation in Sepsis and Systemic Inflammatory Response Syndrome,” Crit. Care Med. 35(9 Suppl.):5584-90 (2007); Rinaldo et al., “Antioxidant Therapy in Critically Septic Patients,” Curr. Drug Targets 10(9):872-80 (2009); Bulger et al., “An Argument for Vitamin E Supplementation in the Management of Systemic Inflammatory Response Syndrome,” Shock 19(2):99-103 (2003), the disclosure of which are incorporated herein by reference in their entirety.

A further aspect relates to a method of treating a patient for SIRS which includes performing a method according to the previous aspects described herein to diagnose the patient with SIRS; and administering a therapy to the patient to treat the SIRS. This method is carried out in accordance with the aspects described supra. Diagnosing a patient with SIRS, therefore, may involve a negative result as to sepsis. Treatment of SIRS can be carried out using the procedures described above.

It is to be appreciated that certain aspects, modes, embodiments, variations and features are described in various levels of detail in order to provide a substantial understanding of the present technology. The definitions of certain terms as used in this specification are also provided. Unless defined otherwise, all technical and scientific terms used herein generally have the same meaning as commonly understood by one of ordinary skill in the art to which this belongs.

### Examples

The following examples are provided to illustrate embodiments of the claimed invention but are by no means intended to limit its scope.

### Materials and Methods for Examples 1-4

Sepsis Mouse Models—

Endotoxemia and CLP were performed according to Animal Resource Protocol approved by the Committee at University of Rochester. For endotoxemia assay, 8-12-wk-old C57BL/6 (Harlan) male mice were weighed and LPS (E. coli O55:B, Sigma-Aldrich) was administered by intra peritoneal (IP) injection to achieve LD90 mortality rate. CLP survival surgery was performed under isofluorane inhalation anesthesia. Caecum was ligated and punctured through and through with a 21-gauge needle. All the mice were resuscitated with 1 ml lactated Ringers injected subcutaneously. For VLA-3 blocking experiments, LXY2 (88 μg) (Cys_{D}-Asp_{D}-Gly-Tyr(3-NO₂)-Gly-4Hyp-Asn-Cys_{D}) where the amino acids may be D amino acids, or control (88 μg) (Peptide International) peptides were administered intravenously (IV) at 2 hours and by IP at 8 hours, 32 hours following CLP or endotoxemia. Survival was monitored for 120 hours.

Generation of Conditional Knockout Animals—

For generation of granulocyte specific integrin knockout mice, Granulocyte Elastase 2 (Ela)-Cre knock-in mice were purchased from The European Mouse Mutant Archive (EMMA), in which Cre-recombinase is expressed in the myeloid cells in place of Ela gene permitting conditional mutagenesis in the myeloid precursors of target genes tagged with loxP sites (Tkalcevic et al., “Impaired Immunity and Enhanced Resistance to Endotoxin in the Absence of Neutrophil Elastase and Cathepsin G,” Immunity 12:201-210 (2000), the disclosure of which is incorporated herein by reference in its entirety). Ela-Cre mice were crossed with and α₃-Flox and αᵥ-Flox mice for four to five generations to achieve deletion of α₃ and αᵥ gene in the transgenic mice. Mice were genotyped by PCR from DNA isolated from tail tissues. For Ela-Cre, primers F (CAT GAC ACC CCC ACT GTC GTG TCC) (SEQ ID NO: 1), R (TGG CAC CAC AGA AAT GAC CTC CAC) (SEQ ID NO: 2) and Lx (TTT GGT GCA CGG TCA GCA GAT TGG) (SEQ ID NO: 3) were used to generate bands of 615 bp and 185 bp for wt and mutant, respectively. For integrin α₃ primers P1 (GAA CAA CAT CTG CCT GGA GT) (SEQ ID NO: 4), and P2 (GTA TGA CTT CTG CCA TGT AGC) (SEQ ID NO: 5) were used to generate bands of 442 bp and 494 bp for wt and flox, respectively. Removal of the gene by Cre-mediated recombination was confirmed using primers P1 and P3 (CAA CAG CAC TGC TGT AGC A) (SEQ ID NO: 6) to generates band of 427 bp (Margadant et al., “Integrin Alpha3beta1 Inhibits Directional Migration and Wound Re-Epithelialization in the Skin,” J. Cell Sci. 122:278-288 (2009), the disclosure of which is incorporated herein by reference in its entirety). For integrin αᵥ primers F (GGT GAC TCA GAC CTT CAG C) (SEQ ID NO:7) and Rev (CAC AAA TCA AGG ATG ACC AAA CTG AG) (SEQ ID NO: 8) were used to generate bands of 550 bp, 700 bp for wild type and floxed mice and 150 bp after Cre-mediated deletion (Lacy-Hulbert et al., “Ulcerative Colitis and Autoimmunity Induced by Loss of Myeloid Alphav Integrins,” Proc. Natl. Acad. Sci. USA 104:15823-15828 (2007), the disclosure of which is incorporated herein by reference in its entirety). For all experiments and further breeding, transgenic mice with brown coat color (Coat color of Ela-Cre parents) were selected.

Isolation and In Vitro Stimulation of Neutrophils—

Blood was collected from healthy volunteers via antecubital vein puncture in heparin containing vacutainers. Granulocytes and erythrocytes were separated from whole blood by centrifugation through 1-step Polymorphs (Fresenius Kabi Norge AS) density gradient. Remaining erythrocytes were removed by hypotonic lysis, yielding a neutrophil purity of >98%. The Human Research Studies Review Board of the University of Rochester approved this study, and informed consent was obtained in accordance with the Declaration of Helsinki To measure integrin expression, neutrophils were stimulated with PMA (20 ng/ml), TNF-α (20 ng/ml), LPS (100 μg/ml) or fMLP (1 μM for 1 hour or 3 hours in L15 (Leibovitz) medium with glucose at 37° C. temperatures.

Sepsis Patient Sample Collection—

Patients admitted to Intensive Care Unit with at least 2 out of 4 SIRS criteria (Temperature >38° C. or <36° C., Heart rate >90/m, Respiratory rate >20 breaths/m or pCO₂<32 mm Hg, leukocyte count >12,000, <4,000, or >10% immature forms on peripheral blood smear) and at least one acute organ dysfunction were enrollment into the study with a requirement that vital abnormalities are confirmed on two occasions. Blood and urine samples were collected within 36 hours of diagnosis and 3-5 days later. Severe sepsis was confirmed by clinical microbiological cultures. Patients with severe non-infectious SIRS met the above SIRS and acute organ dysfunction criteria but did not have documented infection. Blood samples were processed immediately for neutrophil isolation and flow cytometry at 4° C. For measuring surface expression of integrins, purified mouse anti-human integrin α₃, α₅ and αᵥ (Millipore), mouse IgG1 (eBioscience, San Diego, Calif., USA) isotype control and Phycoerythrin (PE) labeled Rat anti-mouse secondary antibodies were used.

Flow Cytometry—

For flow cytometry measurement of the integrin expression on neutrophil, bone marrow (BM), peritoneal Lavage (PL), peripheral blood and lungs were isolated from naïve and septic mice at the indicated time points and subsequently single-cell suspensions were prepared. RBC lysis was performed using ACK lysing buffer (Invitrogen, San Diego, Calif.). The Fc receptors were blocked with unconjugated anti-CD16/32 (eBioscience, San Diego, Calif.) for 30 minutes. Samples were stained with Alexafluor 488 labeled anti-Gr1 (Invitrogen). Allophycocyanin (APC) labeled Ly6G (BD Biosciences), PerCp-Cy5.5 labeled anti-CD11b (eBioscience, San Diego, Calif.,), fluorescein isothiocyanate (FITC) anti-F4/80 (eBioscience, San Diego, Calif., USA), purified goat anti-mouse integrin α₃/CD49c (R&D Systems), PE-anti-mouse integrin α₂/CD49b, α₅/CD49e, αᵥ/CD51, APC-anti-mouse integrin α₆/CD49f and PE conjugated donkey anti-goat IgG (Santa Cruz) antibodies were used. All samples were fixed with 3.7% formaldehyde and collected on FACS Calliber flow cytometer (BD Biosciences, San Diego, Calif.). Data were analyzed using Flow Jo software.

Real-Time PCR—

Total RNA was prepared from human neutrophils or sorted mouse bone marrow granulocytes using RNeasy Mini Kit (QIAGEN, Valencia, Calif.). Similarly, RNA was reverse transcribed by using murine leukemia virus reverse transcriptase (Life Technologies, Bethesda, Md.), with oligo(dT) as the primer (Invitrogen, San Diego, Calif.). All cDNAs were divided into aliquots and stored at −20° C. until further use. Quantitation of integrins α₃ and α₂ on human neutrophils and IL-6, IL-β and TNFα mRNA were performed using TaqMan® Gene Expression Assays (Applied Biosystems). PCR was performed using SYBR green PCR master mix (Applied Biosystems) according to the manufacturer's protocol. PCR amplification of a housekeeping gene, the murine/human hypoxanthine phosphoribosyl transferase gene (GAPDH), was done for each sample as a control for sample loading and to allow normalization between samples. RQ values were analyzed using software.

Multiphoton Intravital Microscopy (MP-IVM)—

For MP-IVM of Lysozyme M (LysM)-GFP mice (Faust et al., “Insertion of Enhanced Green Fluorescent Protein Into the Lysozyme Gene Creates Mice With Green Fluorescent Granulocytes and Macrophages,” Blood 96:719-726 (2000), the disclosure of which is incorporated herein by reference in its entirety) in which granulocytes and monocytes express GFP were used. In order to accurately track morphological changes and migration velocities of individual extravasating leukocytes, mixed bone marrow chimera mice were generated using 80% WT and 20% LysM-GFP bone marrow into irradiated WT recipient mice to reduce the density of fluorescent cells during imaging. 8 weeks after bone marrow transfer to the irradiated recipient mice, the mice were then used for in vivo imaging. Cremaster muscle was prepared for imaging as previously described (Sumagin et al., “Leukocyte-Endothelial Cell Interactions are Linked to Vascular Permeability Via ICAM-1-Mediated Signaling,” Am. J. Physiol. Heart Circ. Physiol. 295:H969-H977 (2008), the disclosure of which is incorporated herein by reference in its entirety). Briefly, mice were anesthetized with pentobarbital sodium (65 mg/kg IP) and maintained on isoflorane inhalation anesthesia. The body temperature was maintained by placing the animal on a warming pad set. The right cremaster muscle was exteriorized and gently pinned over a custom-designed stage for visualization of Cremaster venules by microscopy. Leukocyte extravasation was induced by superfusion of fMLP (1 μM) on to the exteriorized cremaster tissue and time-lapse imaging was performed for 60 minutes using the Olympus FV1000-AOM multiphoton system equipped with 25×NA1.05 water immersion objective (Auffray et al., “Monitoring of Blood Vessels and Tissues by a Population of Monocytes With Patrolling Behavior,” Science 317:666-670 (2007), the disclosure of which is incorporated herein by reference in its entirety). During preparation and observation, the tissue was continuously superfused with warmed bicarbonate-buffered saline. To label blood vessels Texas Red dextran 70,000 MW (20 mg/kg) was injected intravenously into the tail vein (Invitrogen). VLA-3 blocking peptide, LXY2 (88 μg), was injected IV when extravasating cells were noticed. Upon completion of the protocols, the animal was euthanized by anesthetic overdose for labeling neutrophils in VLA-3 conditional knockout (cKO) and Ela-Cre animals, Alexafluor 488 conjugated Gr1 antibody was administered IV in tail vein. Imaging data were analyzed with velocity software (PerkinElmer).

Electron Microscopy—

Leukocyte extravasation was first observed on the fMLP-stimulated cremaster venules of LysM-GFP mice by MP-IVM. LXY2 (88 μg) peptide was injected IV when extravasating cells were noticed. The cremaster was then immediately dissected from the body post-euthanization and fixed with 2.5% glutaraldehyde. The tissue was further processed for transmission electron microscopy at the Electron Microscope Research Core at the University of Rochester for transmission electron microscopy.

Measurement of MPO Activity by Bioluminescence Assay—

For in vitro MPO measurement, sorted neutrophils (5×10⁴ cells/well) were resuspended in buffer containing HEPES and 1% FBS followed by stimulation with 1 μM PMA. 1 μg of luminol salt (5-amino-2-,3-dihydro-1, 4-pthalazinedione) (Sigma, St Louis, Mo.) was added to each well following PMA stimulation. To measure the fluorescence intensity, mice or 96 well plates were imaged with using Xenogen IVIS imaging system (Caliper Lifesciences, Hopkinto, Mass.). Images were analyzed using Living Image 3.2 software. Addition of luminol in the absence of PMA had zero fluorescence.

Bacterial Clearance Assay—

Peritoneal lavage was collected after injection of 10 ml PBS into the peritoneum. 1:100 1:1000 and 1:10000 dilution was performed and 100 μl from each dilution was spread on tryptic Soy Agar (TSA) Blood Agar Plates (Remel, Lenexa, Kans.). All the plates were incubated at 37° C. temperatures for 24 hours. Colony count for each group was expressed as CFU/ml.

Phenotype of Gr1⁺ Mouse Neutrophils—

Cells were isolated from peritoneal lavage of septic mice and were stained with FITC labeled anti-Gr1 antibody and sorted into Gr1^{high} and Gr1^{low} granulocytes with FACS Aria (BD Biosciences, San Diego, Calif.). Cells were spun down on a slide using Shandon cytospin-2 followed by hematoxylin and eosin staining Morphology of the cells was analyzed under 20× magnification.

IL-6 Cytokine ELISA—

Serum from individual mice was collected at the indicated time points and the level of IL-6 was determined by standard sandwich ELISA protocol. Anti-IL-6, capture and detection Abs were purchased from BD Pharmingen and standard recombinant murine IL-6 and Strepravidin-HRP were purchased from PeproTech (Rocky Hill, N.J.) and thermo scientific respectively. The color reaction was developed using TMB (Thermo Scientific, Rockford, Ill.) and measured with an ELISA reader (Kinetic microplate reader; Molecular Devices) at 450 nm.

Data Analysis—

All values are expressed as the mean±SEM. Data from human samples were analyzed by one-way ANOVA. Data from murine experiments were analyzed by paired non-parametric t-test (Wilcoxon test). Survival curves were analyzed by the Kaplan-Meyer log-rank test. All statistics were performed using the GraphPad Prism 4.0 software. P value <0.05 was considered significant.

### Example 1

### VLA-3 Expression is Increased in Septic Neutrophils

The dynamic migration of neutrophils first through the endothelium and subsequently through the extracellular matrix is involved in the pathophysiology of sepsis-associated tissue injury and organ dysfunction (Brown et al., “Neutrophils in Development of Multiple Organ Failure in Sepsis,” Lancet 368:157-169 (2006), the disclosure of which is incorporated herein by reference in its entirety). Although β₂ (CD18) integrins, including LFA-1 (α_{L}β₂; CD11a/CD18) and Mac-1 (α_{M}β₂; CD11b/CD18), have been shown to be upregulated on septic neutrophils and to mediate transendothelial migration (TEM) during systemic and local inflammation (Lin et al., “Altered Leukocyte Immunophenotypes in Septic Shock. Studies of HLA-DR, CD11b, CD14, and IL-2R expression,” Chest 104:847-853 (1993), the disclosure of which is incorporated herein by reference in its entirety), relatively little information is available concerning the ability of neutrophils to control migration in interstitial tissue.

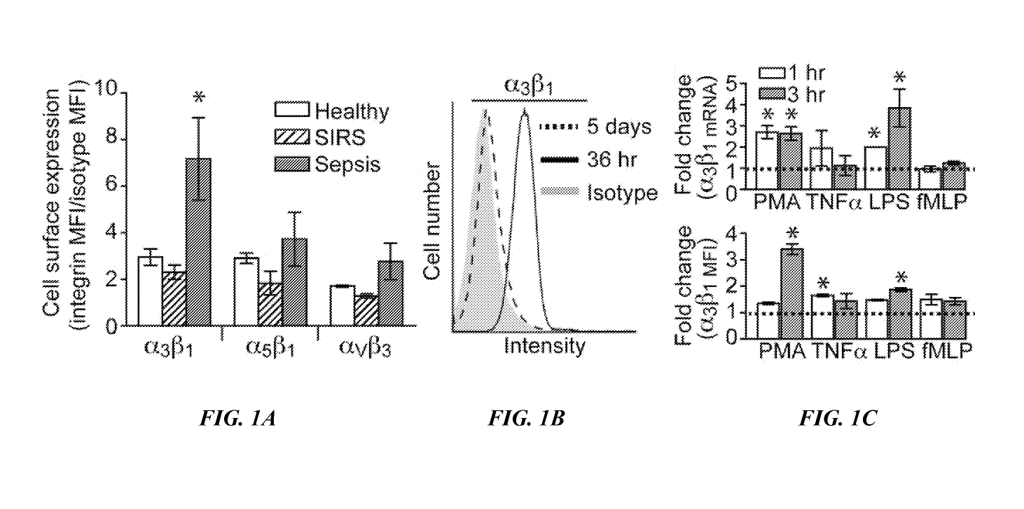

Neutrophils express several cell-surface integrins that can bind to ECM proteins (Lindbom et al., “Integrin-Dependent Neutrophil Migration in Extravascular Tissue,” Semin. Immunol. 14:115-121 (2002), the disclosure of which is incorporated herein by reference in its entirety). To determine which ECM binding integrin is important for neutrophil trafficking during sepsis, circulating neutrophils were harvested from patients with severe sepsis, patients with non-infectious severe SIRS (systemic inflammatory response syndrome), and healthy volunteers. Unlike other integrins, the surface expression levels of VLA-3 on neutrophils from patients with severe sepsis were significantly elevated as compared to the healthy control subjects. Interestingly, up regulation of VLA-3 level was not observed in subjects with severe non-infectious SIRS (FIG. 1A), demonstrating that VLA-3 is a novel cell surface marker that can discriminate sepsis from SIRS. As shown in FIG. 1B, VLA-3 expression was elevated within 24-36 hours of diagnosis (n=6), and the levels returned to normal upon recovery (n=4). The increases in the mRNA and cell-surface levels of VLA-3 were further investigated in neutrophils isolated from the peripheral blood of healthy subjects and incubated with various stimulators. Neutrophil activation by PMA and LPS induced significant increases in both the mRNA and protein levels of VLA-3 within 1 and 3 hours of stimulation (FIG. 1C).

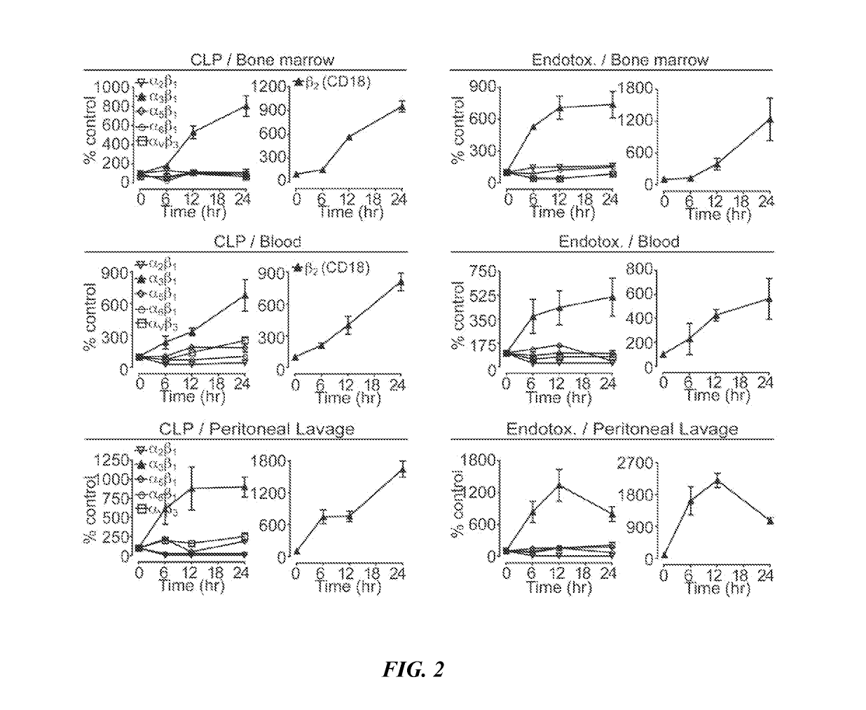

To perform detailed measurement of the expression kinetics of VLA-3 and other ECM protein-binding integrins during sepsis, the following two mouse models were used: LPS-induced endotoxemia and cecal ligation and puncture (CLP). In both models, neutrophil stimulation was confirmed by the elevated surface expression of CD18, a well-known marker of neutrophil activation (FIG. 2). Flow cytometry analysis of neutrophils isolated from the bone marrow, peripheral blood, and peritoneal lavage of endotoxemia and CLP mice revealed that of the ECM protein-binding integrins analyzed, only VLA-3 expression was dramatically enhanced (FIG. 2). The proportion of VLA-3 expressing neutrophils in the circulation also increased following onset of sepsis in both CLP and endotoxemia induced peritonitis. The results presented herein demonstrate that the expression of VLA-3 on neutrophils is enhanced during systemic inflammation, and this finding may be useful for the recruitment of neutrophils into peripheral tissues during sepsis.

### Example 2

### Gr1^{high}CD11b^{high}VLA-3^{high} Septic Neutrophil Subpopulation Exhibits Pro-Inflammatory Phenotypes

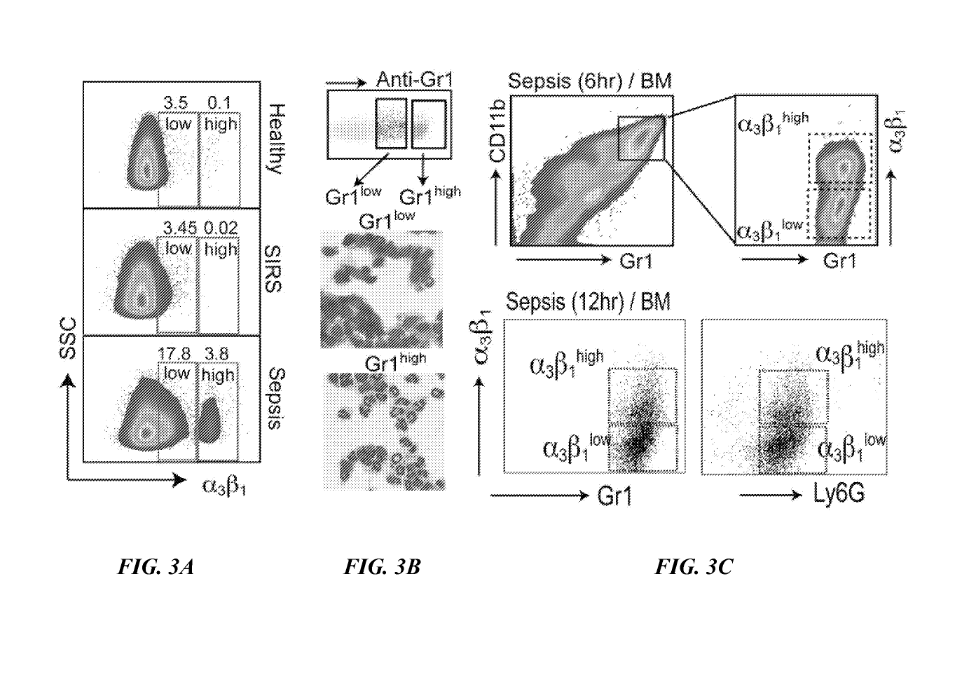

Interestingly, a significant increase in VLA-3 expression was more evident in a subpopulation of the active neutrophils isolated from sepsis patients (FIG. 3A). This VLA-3^{high} neutrophil population was not seen in SIRS patients or healthy subjects. Similar to human patients, among all active neutrophils (Gr1^{high}CD11b^{high}) from septic mice, only a subpopulation of cells expressed higher levels of VLA-3 (FIGS. 3B and C). This was confirmed using both the Gr1 and Ly6G antibodies against neutrophil specific markers (FIG. 3C, Lower panel).

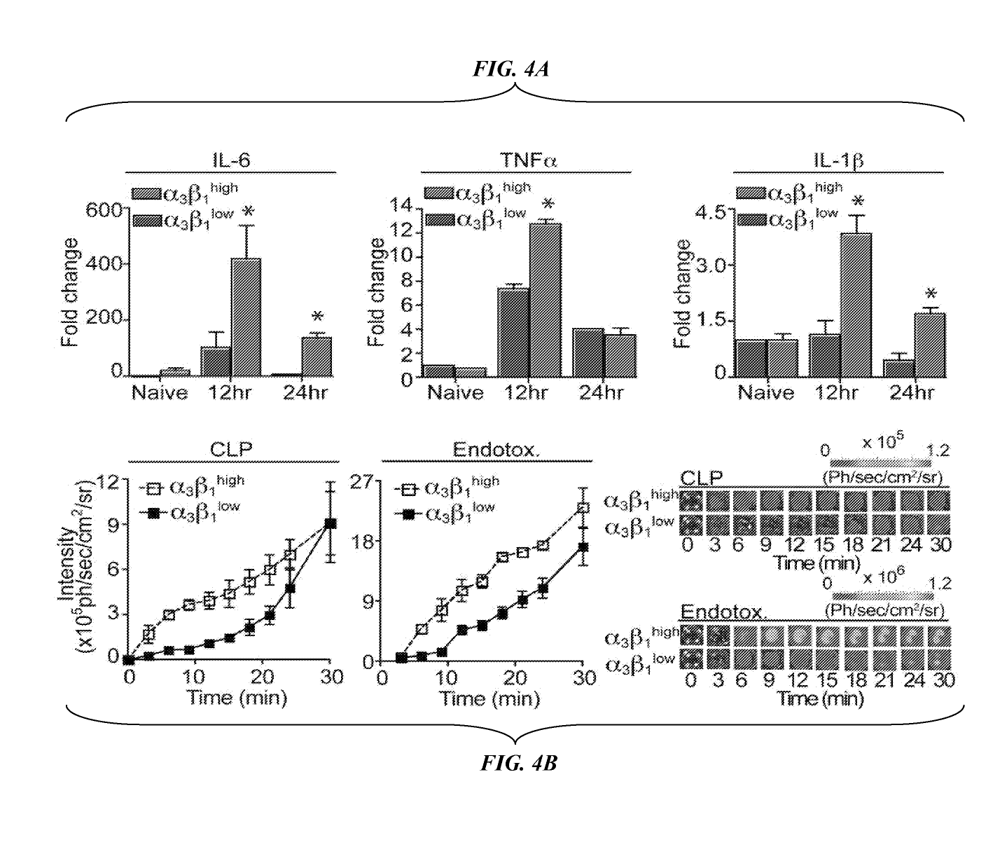

It has previously been proposed that a hyper-inflammatory neutrophil subtype arises during sepsis, which may cause tissue damage and lead to MOF in sepsis (Brown et al., “Neutrophils in Development of Multiple Organ Failure in Sepsis,” Lancet 368:157-169 (2006), the disclosure of which is incorporated herein by reference in its entirety). Increased systemic release of pro-inflammatory cytokines (such as IL-6, tumor necrosis factor-α (TNF-α) and IL-1β) and elevated MPO activity are biomarkers of hyper-responsive neutrophils (Nathan, C., “Neutrophils and Immunity: Challenges and Opportunities,” Nat. Rev. Immunol. 6:173-182 (2006), the disclosure of which is incorporated herein by reference in its entirety). To determine whether the Gr1^{high}CD11b^{high}VLA-3^{high} cells are associated with a hyper-inflammatory phenotype, the mRNA levels of pro-inflammatory cytokines were measured. In CLP mice, the expression levels of IL-6, TNF-α and IL-1β mRNA were significantly increased in the Gr1^{high}CD11b^{high}VLA-3^{high} neutrophils, but the levels of pro-inflammatory cytokines in the Gr1 CD11b^{high}VLA-3^{low} population were similar to the levels in naïve Gr1 CD11b^{high}VLA-3^{low} neutrophils (FIG. 4A). Next, the enzymatic activity of MPO in the two populations of neutrophils (Gr1^{high}CD11b^{high}VLA-3^{high} vs. Gr1^{high} CD11b^{high}VLA-3^{low}) was measured. As shown in FIG. 4B, the MPO activity of the Gr1^{high}CD11b^{high}VLA-3^{high} neutrophils from both the CLP and endotoxemia mice exhibited a higher sensitivity to PMA stimulation than the Gr1^{high}CD11b^{high}VLA-3^{low} cells. Taken together, the data presented herein demonstrates that VLA-3 is a novel biomarker for a septic neutrophil subset with hyper-inflammatory phenotypes.

### Example 3

### Blocking and Conditional Depletion of VLA-3 Improves Survival During Sepsis by Interfering with Neutrophil Extravasation and Migration

Blocking the recruitment of inflammatory neutrophils from the vasculature into tissues has been proposed as a treatment for severe systemic inflammation (Grommes et al., “Contribution of Neutrophils to Acute Lung Injury,” Mol. Med. 17:293-307 (2011); Souto et al., “Essential Role of CCR2 in Neutrophil Tissue Infiltration and Multiple Organ Dysfunction in Sepsis,” Am. J. Respir. Crit. Care Med. 183:234-242 (2011), the disclosure of which are incorporated herein by reference in their entirety). However, in extreme situations, this approach could mimic leukocyte adhesion deficiency, a frequently fatal set of syndromes that impair the host defense against infection. Therefore, the general inhibition of neutrophil extravasation or TEM could prove fatal by leaving all activated neutrophils within the circulation. Interestingly, partial depletion of CD18 was shown to improve inflammation (Wilson et al., “Gene Targeting Yields a CD18-Mutant Mouse for Study of Inflammation,” J. Immunol. 151:1571-1578 (1993), the disclosure of which is incorporated herein by reference in its entirety).

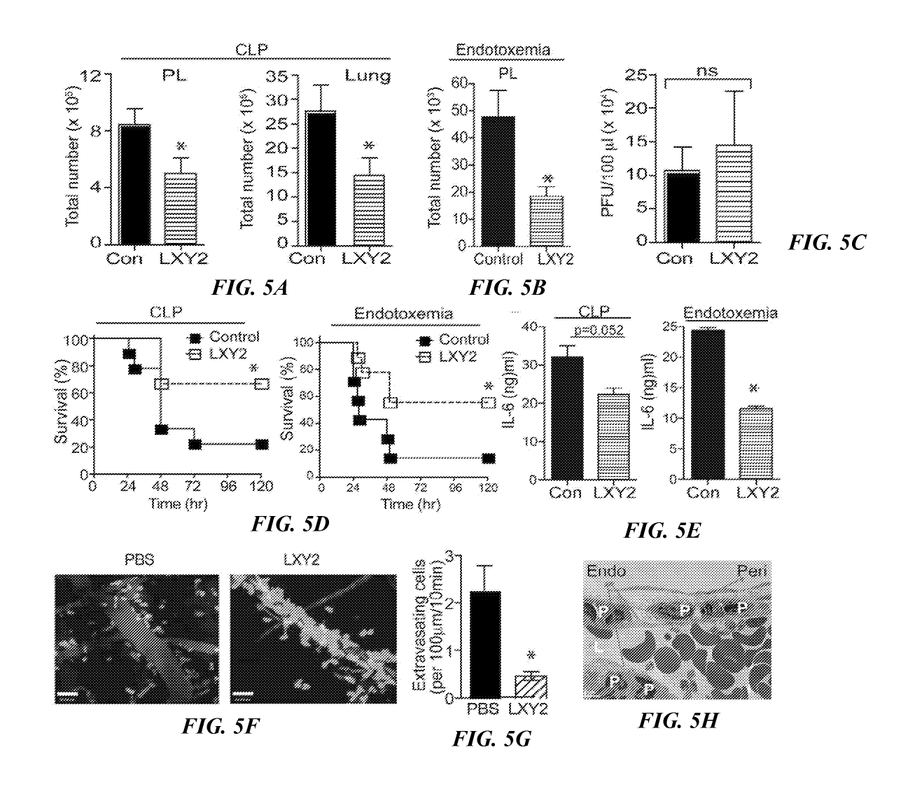

It was next assessed whether selectively targeting the VLA-3^{high} hyper-inflammatory neutrophil subset and blocking their infiltration during sepsis is beneficial in reducing the severity of multi-system organ dysfunction and failure and mortality in sepsis. Administration of the VLA-3 small molecule antagonist peptide LXY2 (Yao et al., “Discovery of Targeting Ligands for Breast Cancer Cells Using the One-Bead One-Compound Combinatorial Method,” J. Med. Chem. 52:126-133 (2009), the disclosure of which is incorporated herein by reference in its entirety) significantly reduced the number of tissue-infiltrating neutrophils in mice with CLP (FIG. 5A) and endotoxemia (FIG. 5B) induced sepsis without altering local bacterial clearance (FIG. 5C). Similarly, LXY2 protected mice from sepsis lethality (FIG. 5D) and reduced the levels of the IL-6 cytokine in the serum (FIG. 5E).

To access the inflamed tissues, neutrophils must cross the basement membrane, which is an ECM structure underlying the vasculature. VLA-3 has been implicated in neutrophil chemotaxis along ECM proteins, including fibronectin and laminin, which are the predominant components of the basement membrane (Elices et al., “Receptor Functions for the Integrin VLA-3:Fibronectin, Collagen, and Laminin Binding are Differentially Influenced by Arg-Gly-Asp Peptide and by Divalent Cations,” J. Cell Biol. 112:169-181 (1991); Harler et al., “Promotion of Neutrophil Chemotaxis Through Differential Regulation of Beta 1 and Beta 2 Integrins,” J. Immunol. 162:6792-6799 (1999), the disclosure of which are incorporated herein by reference in their entirety). MP-IVM of the blood vessels revealed that the treatment of mice with LXY2 dramatically reduced neutrophil extravasation in response to fMLP (FIGS. 5F and 5G). Additionally, ultrastructural analyses showed that the majority of neutrophils accumulated within the space between the endothelium and the basement membrane (FIG. 5H), indicating that the interaction between VLA-3 and its ligands is involved in the extravasation of the hyper-responsive neutrophils under inflammatory conditions.

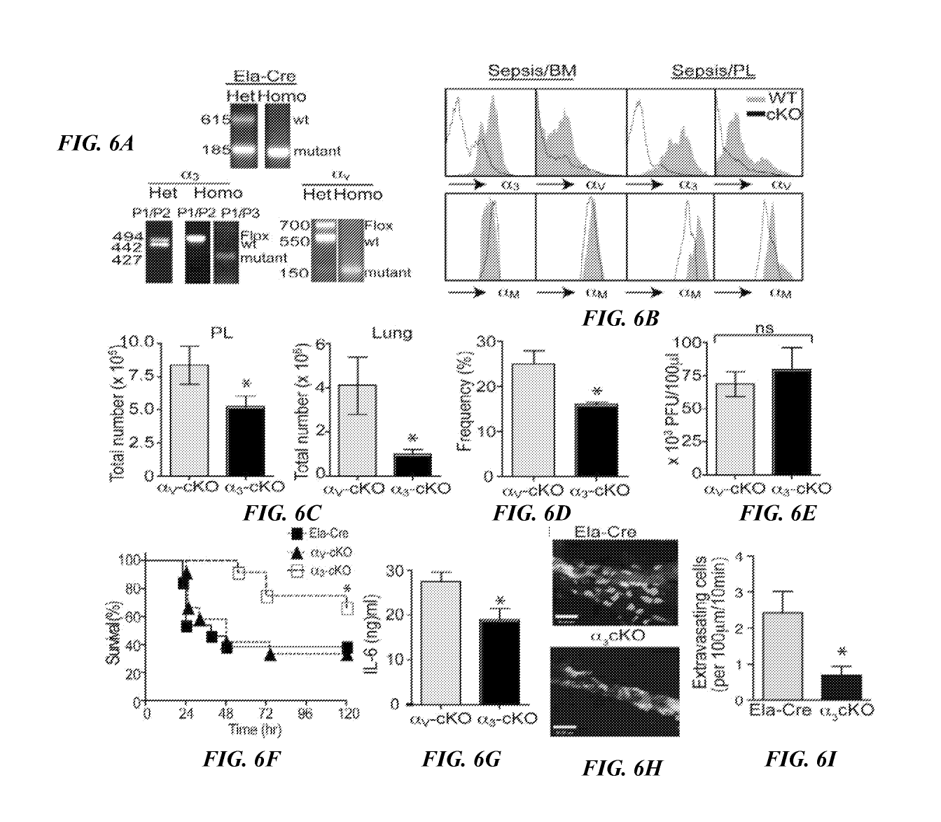

Determining the precise contributions of VLA-3 to immune functions in vivo has been challenging due to the lethality of VLA-3 knockout in mice, which die from developmental defects of the kidney and lung (Kreidberg et al., “Alpha 3 Beta 1 Integrin Has a Crucial Role in Kidney and Lung Organogenesis,” Development 122:3537-3547 (1996), the disclosure of which is incorporated herein by reference in its entirety). To circumvent this problem and to further assess the function of VLA-3 in sepsis, a conditional knockout of VLA-3 (α₃-cKO) was generated by crossing α3-flox mice with mice expressing Cre under the granulocyte elastase promoter (Ela-Cre). No increase in the cell-surface expression of αᵥ integrin was detected on septic neutrophils (FIG. 2D). Therefore, a conditional knockout of αᵥ integrin (αᵥ-cKO) was also generated as a control. These cKO mice were born at the expected frequencies and developed normally without evidence of defects. The Ela-Cre transgenic mice express Cre in granulocytes, leading to gene deletion specifically in neutrophils. Both the deletion of the floxed a subunit alleles and absence of the proteins were confirmed (FIGS. 6A and 6B). Compared to the Ela-Cre and αᵥ-cKO mice, the depletion of VLA-3 dramatically reduced both the number and frequency of neutrophils infiltrating the peritoneum and lungs within 6 h of CLP (FIGS. 6C and 6D). Survival during sepsis was significantly improved, but bacterial clearance was unaffected (FIGS. 6E and 6F). Additionally, levels of IL-6 were significantly reduced in the α₃-cKO mice compared to αᵥ-cKO mice, indicating reduced severity of sepsis (FIG. 6G). To measure the effect of VLA-3 removal of neutrophil extravasation, MP-IVM was performed in the inflamed cremaster muscle vasculature. Supporting the results described herein, the number of extravasating neutrophils was significantly reduced in the of α₃-cKO mice as compared to control animals (FIGS. 6H and 6I). Taken together, the data presented herein demonstrates that the selective depletion of the integrin VLA-3 from granulocytes successfully decreases the massive tissue infiltration of neutrophils and improves survival during sepsis.

### Discussion of Examples 1-3

The dynamic migration of neutrophils first through the endothelium and subsequently through the ECM is involved in the pathophysiology of sepsis-associated tissue injury and organ dysfunction (Brown et al., “Neutrophils in Development of Multiple Organ Failure in Sepsis,” Lancet 368:157-169 (2006); Kovach et al., “The Function of Neutrophils in Sepsis,” Curr. Opin. Infect. Dis. 25(3):321-7 (2012), the disclosure of which are incorporated herein by reference in their entirety). Following diapedesis across the capillary endothelium, neutrophils must cross the subendothelial basement membrane to migrate through the ECM and gain access to the tissue space (Nourshargh et al., “Breaching Multiple Barriers: Leukocyte Motility Through Venular Walls and the Interstitium,” Nat. Rev. Mol. Cell Biol. 11:366-378 (2010), the disclosure of which is incorporated herein by reference in its entirety). VLA-3 mediates neutrophil chemotaxis through the basement membrane (Hyun et al., “Uropod Elongation is a Common Final Step in Leukocyte Extravasation Through Inflamed Vessels,” J. Exp. Med. 209(7):1349-62 (2012), the disclosure of which is incorporated herein by reference in its entirety). In the preceding Examples, it is shown that integrin VLA-3 is associated with massive neutrophil infiltration during sepsis, and selective blocking of VLA-3 on neutrophils inhibits their migration through the basement membrane.

As the first line of immune defense, neutrophils are often considered to be a terminally differentiated and homogeneous cell population. During inflammation, however, neutrophils actively migrate into infected or inflamed tissues and present themselves to local inflammatory mediators that prolong their survival and retention during disease progression (Colotta et al., “Modulation of Granulocyte Survival and Programmed Cell Death by Cytokines and Bacterial Products,” Blood 80:2012-2020 (1992), the disclosure of which is incorporated herein by reference in its entirety). As described herein, several independent lines of evidence are presented to support the existence of a previously unrecognized VLA-3^{high}/hyper-inflammatory subpopulation of neutrophils during sepsis. First, flow cytometry analysis of circulating and tissue-infiltrating neutrophils from septic human patients and murine sepsis models revealed that a subpopulation of activated neutrophils expresses high levels of cell-surface VLA-3. Second, the VLA-3^{high} neutrophil subpopulation disappeared upon recovery from sepsis in human patients. Third, the dramatic upregulation of VLA-3 expression during sepsis was associated with increased pro-inflammatory cytokine expression, elevated MPO activity, and rapid tissue infiltration by neutrophils. Finally, the administration of a specific peptide inhibitor of VLA-3 and the selective genetic depletion of VLA-3 in neutrophils both improved the survival of septic mice. Consistent with the findings described herein, emerging evidence has demonstrated the existence of functionally heterogeneous neutrophil subsets, including those that exhibit differential expression of fMLP (al-Essa et al., “Heterogeneity of Circulating and Exudated Polymorphonuclear Leukocytes in Superoxide-Generating Response to Cyclic AMP and Cyclic AMP-Elevating Agents. Investigation of the Underlying Mechanism,” Biochem. Pharmacol. 49:315-322 (1995), the disclosure of which is incorporated herein by reference in its entirety) and T-cell receptors (Puellmann et al., “A Variable Immunoreceptor in a Subpopulation of Human Neutrophils,” Proc. Natl. Acad. Sci. USA 103:14441-14446 (2006), the disclosure of which is incorporated herein by reference in its entirety). In addition, two subsets of neutrophils with distinct cytokine and chemokine production profiles, different effects on macrophage activation, and unique surface antigen expression have been identified in mice (Tsuda et al., “Three Different Neutrophil Subsets Exhibited in Mice with Different Susceptibilities to Infection by Methicillin-Resistant Staphylococcus aureus,” Immunity 21:215-226 (2004), the disclosure of which is incorporated herein by reference in its entirety) and humans (Chakravarti et al., “Reprogramming of a Subpopulation of Human Blood Neutrophils by Prolonged Exposure to Cytokines,” Lab Invest. 89:1084-1099 (2009), the disclosure of which is incorporated herein by reference in its entirety). The findings described herein thus add a novel aspect to the current understanding of neutrophil function in the mammalian immune system.

Whereas activated neutrophils can damage tissue, they can also kill bacteria. The non-selective suppression of stimulated neutrophils thus may not benefit patients with severe sepsis and could adversely affect host defenses. Antibodies directed against adhesion molecules important for leukocyte extravasation, such as β₂ (CD18) integrins (LFA-1 and Mac-1) and ICAM-1 (their endothelial membrane counterpart ligands), have shown benefit in certain settings but have been associated with devastating infections. Because life-threatening infections generally appear much sooner in neutropenic conditions than in lymphopenic patients, the administration of agents that block the recruitment of neutrophils into infected tissue sites has a greater risk of infection and immune response impairment compared to that seen upon the use of agents that interfere with lymphocyte recruitment. Therefore, a major challenge in treating sepsis is the lack of therapeutic strategies to manipulate immune functions only in the aberrantly activated leukocytes. Increased expression of VLA-3 was observed in the circulation and in tissues during sepsis, and this upregulation of VLA-3 correlated with exaggerated neutrophilic inflammation, organ damage, and lethality. These results demonstrate that VLA-3 is a novel marker for hyper-responsive and pro-inflammatory neutrophils and is a suitable target for anti-inflammatory therapy directed against aberrantly activated neutrophils during sepsis. Consistently, the administration of a VLA-3 antagonist as well as the conditional depletion of VLA-3 in neutrophils reduced number of infiltrating neutrophils in the lungs of septic mice and improved survival.

Previously, a study by Werr et al. (Werr et al., “Beta1 Integrins are Critically Involved in Neutrophil Locomotion in Extravascular Tissue In Vivo,” J. Exp. Med. 187:2091-2096 (1998), the disclosure of which is incorporated herein by reference in its entirety) has shown that cell surface expression of β₁ integrin is highly upregulated on extravasated PMNs during inflammation and that members of the β1 integrin subfamily other than VLA-4 (α₄β₁) and VLA-5 (α₅β₁) are important for extravascular PMN motility. As demonstrated herein, there was a significant difference in VLA-3 expression levels in SIRS and severe sepsis patients. SIRS is commonly seen after major surgery or trauma and involves severe inflammation. If SIRS is accompanied by a confirmed source of infection, it is diagnosed as sepsis. Thus, these results indicate that strong TLR ligand-mediated effects of microbial components in addition to the pro-inflammatory milieu could be involved in VLA-3 upregulation on neutrophils during sepsis. Because the prompt diagnosis of sepsis warrants rapid therapy, including the early administration of antibiotics and control of the source of sepsis, the accurate differentiation between sepsis and SIRS is a significant first step leading to effective therapy. These data demonstrate that VLA-3 is of value for differentiating between sepsis and SIRS in critically ill patients, and also represents a therapeutic target for controlling host tissue damage induced by a VLA-3^{high} subset of activated neutrophils.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1.-15. (canceled)

16. A method of diagnosing sepsis or sepsis risk in a subject, said method comprising:
contacting a biological sample from a subject with a reagent that binds specifically to integrin VLA-3 (CD49c/CD29) in the biological sample;
detecting the reagent bound to integrin VLA-3 in the biological sample, and
determining the expression level of integrin VLA-3 in the biological sample wherein an elevated level of integrin VLA-3 in the biological sample, relative to a control level of integrin VLA-3, indicates that the subject has sepsis or is at risk of developing sepsis.

17. The method according to claim 16, wherein said detecting is carried out using flow cytometry.

18. The method according to claim 16, wherein the biological sample is selected from blood, plasma, serum, or bone marrow.

19. The method according to claim 16, wherein the biological sample is a blood sample.

20.-21. (canceled)

22. The method according to claim 16, wherein the reagent is a monoclonal antibody, or binding fragment thereof, aptamer, or antibody mimic.

23. The method according to claim 16, wherein the integrin VLA-3 is present on neutrophils.

24. The method according to claim 16 further comprising:
contacting the biological sample with a reagent that binds specifically to a neutrophil-specific marker.

25. The method according to claim 24, wherein the neutrophil-specific marker is CD16+CD62L+.

26. The method according to claim 16, wherein the subject is an individual who previously had sepsis and said detecting is used to determine risk of reoccurrence of sepsis in the subject.

27. The method according to claim 16, wherein the subject is an individual with a predisposition to sepsis and said method is used for early detection of the sepsis.

28. (canceled)

29. A method of treating a patient for sepsis comprising:
detecting the presence of a subpopulation of neutrophils having an elevated integrin VLA-3 (CD49c/CD29) expression level in the patient, whereby the presence of the subpopulation of neutrophils indicates that the patient has sepsis or a risk of sepsis; and
administering a therapy to treat the patient for sepsis.

30. The method according to claim 29, wherein the therapy for the subject comprises administration of an integrin VLA-3 antagonist peptide or an anti-VLA-3 antibody.

31. The method according to claim 29, wherein the therapy for the patient comprises depletion of integrin VLA-3 expression.

32. The method according to claim 29, wherein the therapy for the patient comprises a drug, therapy, surgery, or any combination thereof.

33. The method according to claim 32, wherein the drug is an agent for managing sepsis resistance, a blocker of integrin VLA-3, an antibiotic, or a vasopressor.

34. The method according to claim 32, wherein the therapy is selected from the group consisting of oxygen administration, fluid administration, and dialysis.

35.-36. (canceled)

37. The method according to claim 29 further comprising repeating said detecting after said administering the therapy,
wherein when the baseline integrin VLA-3 level is greater than the post-therapy integrin VLA-3 level, the therapy is effective; and when the baseline integrin VLA-3 level is less than the post-therapy integrin VLA-3 level, the therapy is not effective.

38.-41. (canceled)

42. A method of discriminating between sepsis and systemic inflammatory response syndrome (SIRS) comprising:
detecting the presence or absence of a subpopulation of neutrophils having an elevated integrin VLA-3 expression level in a subject having systemic inflammation, whereby the presence of the subpopulation of neutrophils indicates that the subject has sepsis and the absence of the subpopulation of neutrophils indicates that that subject has SIRS.

43. The method according to claim 42 further comprising:
selecting a patient exhibiting one or more clinical symptoms of sepsis and/or SIRS prior to said detecting.

44.-51. (canceled)

52. The method according to claim 42 further comprising: administering to the subject a therapy for SIRS or a therapy for sepsis, depending on the result of said detecting.
